(19) United States
(12) Patent Application Publication
(10) Pub. No.: US 2014/0194346 A1
(43) Pub. Date: Jul. 10, 2014

(54) PASTEURELLACEAE VACCINES

(71) Applicants: Markus Aebi, Wettingen (CH); Flavio Schwarz, Smarano (IT); Andreas Naegeli, Zurich (CH)

(72) Inventors: Markus Aebi, Wettingen (CH); Flavio Schwarz, Smarano (IT); Andreas Naegeli, Zurich (CH)

(73) Assignee: ETH ZURICH, Zurich (CH)

(21) Appl. No.: 14/237,377

(22) PCT Filed: Aug. 7, 2012
(86) PCT No.: PCT/EP2012/065480

§ 371 Date: Feb. 6, 2014

(30) Foreign Application Priority Data
Aug. 8, 2011 (EP) 11006498.7

(51) Int. Cl. C07K 14/285; G01N 33/569

(52) U.S. Cl. CPC C07K 14/285; G01N 33/56911
USPC 514/2.8; 530/395; 435/69.1; 435/7.92

(57) ABSTRACT

The present invention relates to an N-glycosylated protein for treating and/or preventing bacterial Pasteurellaceae infection in a mammal or bird, wherein the protein is a Pasteurellaceae protein, a functional fragment or derivative thereof having at least one glycosylated N-X-S/T consensus sequence. In addition, the present invention is directed to corresponding pharmaceutical compositions for treating and/or protecting mammals or birds having or being prone to develop a bacterial Pasteurellaceae infection. Furthermore, the invention describes methods for producing said N-glycosylated proteins.

The present invention relates to an N-glycosylated protein for treating and/or preventing bacterial Pasteurellaceae infection in a mammal or bird, wherein the protein is a Pasteurellaceae protein, a functional fragment or derivative thereof having at least one glycosylated N-X-S/T consensus sequence. In addition, the present invention is directed to corresponding pharmaceutical compositions for treating and/or protecting mammals or birds having or being prone to develop a bacterial Pasteurellaceae infection. Furthermore, the invention describes methods for producing said N-glycosylated proteins.

### BACKGROUND OF THE INVENTION

Pasteurellaceae comprise a large and diverse family of Gram-negative proteobacteria comprising the genera Actinobacillus, Aggregatibacter, Avibacterium, Basfia, Bibersteinia, Chelonobacter, Gallibacterium, Haemophilus, Histophilus, Lonepinella, Mannheimia, Nicoletella, Pasteurella, Phocoenobacter and Volucribater. Some species presented in these genera are important human or animal pathogens, e.g. Haemophilus influenzae or Mannheimia haemolytica, while others are commensals of the animal and human mucosa, mostly in the upper respiratory tract. H. influenzae causes several respiratory diseases in humans and is also known as an agent of meningitis in children. Other Pasteurellaceae cause gingivitis and chancroid in humans and many others are important veterinary pathogens, e.g. Mannheimia haemolytica, the cause of bronchopneumonia in cattle, Actinobacillus pleuropneumoniae, a responsible agent of pneumonia in sheep, or Haemophilus parasuis causing severe polyserositis in pigs.

The glycosylated Haemophilus influenzae HMW1 adhesin mediates adherence to respiratory epithelial cells, a critical early step in the pathogenesis of H. influenzae disease. All of the glycosylated sites in HMW1 are asparagine residues. The glycosylating enzyme is a protein called HMW1C which transfers glucose and galactose and also generates hexose-hexose bonds. The Actinobacillus pleuropneumoniae protein ApHMW1C shares high-level homology with HMW1C. ApHMW1C has N-glycosyltransferase activity and transfers glucose and galactose to asparagine sites in protein HMW1. In addition, ApHMW1C can complement a deficiency of HMW1C and mediate HMW1 glycosylation and adhesive activity in whole bacteria. So far, there is no evidence for glycosylation of proteins in A. pleuropneumoniae (Choi et al., PLoS ONE 5(12): e15888. doi:10.1371/journal.pone.0015888, 2010).

A phage expression library of the A. pleuropneumoniae genome was screened to identify potential vaccine components. Open reading frames within immuno-reactive phage were analysed in silico to identify conserved outer membrane proteins, four of which, i.e. comL, IolB, IppC and ompA, were antigenic, highly conserved, outer membrane, in vivo-expressed proteins. However, despite a detectable specific antibody response, none of these proteins proved individually capable of protecting pigs from colonization and infection with the homologous A. pleuropneumoniae strain in pig protection studies (Oldfield et al., Vaccine 26, 1942-195, 2008).

In pig protection studies a detectable specific antibody response to recombinant AasP, a subtilisin-like serine protease and a conserved outer membrane-localised autotransporter protein of A. pleuropneumoniae was induced. However, the vaccine proved non-capable of protecting pigs from colonization, infection or severe clinical disease resulting from challenge with the homologous A. pleuropneumoniae strain (Oldfield et al., Vaccine 38:5278-83, 2009).

Porcine contagious pleuropneumonia is caused by A. pleuropneumoniae infection and contributes to major economic losses in the livestock industry. With the need to reduce the use of antibiotics in agricultural livestock, vaccination has emerged as a safer and more cost-effective approach for disease control. Based on surface polysaccharides, fifteen serotypes of A. pleuropneumoniae are differentiated (Blackall et al., Vet Microbiol 84, 47-52, 2002). The many serotypes have made effective vaccination difficult. Killed whole-cell vaccines reduce mortality but only provide serotype-specific immunity (Nielsen, Nord Vet Med 36, 221-234, 1984). However, a natural or experimental infection with one serotype generally provides protection against heterologous challenge, suggesting the existence of common antigens that are cross-protective and exposed during infection, but that are not retained in killed whole-cell preparation (Haesebrouk et al., Vet Microbiol 52, 277-284, 1996). Various purified antigens including Apx toxins (Devenish et al., Infect Immun 58, 3829-3832, 1990), lipopolysaccharide (Rioux et al., Res Vet Sci 65, 165-167, 1998) and outer membrane proteins such as OmlA (Gerlach et al., Infect Immun 61, 565-572, 1993), TbpA (Rossi-Campos et al., Vaccine 10, 512-518, 1992), AasP (Oldfield et al., Vaccine 27, 5278-5283, 2009), ComL, LolB, LppC and OmpA (Oldfield et al., Vaccine 26, 1942-1954, 2008) have also been investigated as vaccine candidates. In general, immunization with single proteins conserved among different serotypes results in a specific but weakly protective immune response. Apx toxins-based vaccines can reduce clinical symptoms but offer only partial protection, indicating that an effective cross-protective vaccine might require several antigens. The current commercial vaccine Porcilis APP (Intervet; now Merck Animal Health) includes a 42-kDa outer membrane protein and the three toxoids ApxI, ApxII and ApxIII produced by A. pleuropneumoniae strains. This product is considered effective for preventing acute disease but does not preclude colonization and is not widely cross protective (Tumamao et al., Aust Vet J 82, 773-780, 2004a, 2004b).

Other vaccines on the market against species from the family Pasteurellaceae include “Bovigrip” (Intervet; now Merck Animal Health), a cattle vaccine which is partially comprised of inactivated Mannheimia haemolytica. This species is also contained in the vaccines “Pastobov” (Merial) and “RispovalPasteurella” (Pfizer).

In view of the above it is the objective of the present invention to provide vaccines for treating and/or preventing infections caused by members of the family Pasteurellaceae, in particular infections caused by members of the genera Haemophilus, Histophilus, Mannheimia and Actinobacillus, which is preferably effective in preventing acute disease, precludes colonization and which is widely cross-protective among different serotypes. In particular, it is the objective of the present invention to provide a safe, cost-effective and efficient vaccine for treating and/or preventing Actinobacillus pleuropneumonia, Mannheimia haemolytica, Haemophilus parasuis and Histophilus somni infections in mammals and birds, in particular in livestock and pet animals. In addition, it is the objective of the present invention to provide a method for diagnosing Pasteurellaceae infections in mammals and birds.

The above objects are solved by the provision of an N-glycosylated protein for treating and/or preventing bacterial Pasteurellaceae infection in a mammal or bird, wherein the protein is a Pasteurellaceae protein, a functional fragment or derivative thereof having at least one glycosylated N-X-S/T consensus sequence, wherein X is not proline.

In a preferred embodiment the Pasteurellaceae protein, functional fragment or derivative thereof is selected from the group of Pasteurellaceae proteins of Actinobacillus, Aggregatibacter, Avibacterium, Basfia, Bibersteinia, Chelonobacter, Gallibacterium, Haemophilus, Histophilus, Lonepinella, Mannheimia, Nicoletella, Pasteurella, Phocoenobacter and Volucribater, preferably proteins of Actinobacillus, Histophilus, Haemophilus, Mannheimia, more preferably proteins from Actinobacillus pleuropneumonia, Haemophilus parasuis, Histophilus somni and Mannheimia haemolytica.

In principle, any protein comprising one or more consensus sequences N-X-S/T, wherein X is not proline, is suitable for being N-linked to a glycan featuring mono-, oligo- or polysaccharides. In eukaryotes, this connection of protein and saccharide at the consensus sequence is facilitated by the enzyme complex oligosaccharyltransferase in the endoplasmic reticulum (for a review see Mohorko et al., J Inherit Metab Dis. 2011 August; 34(4):869-78) while in bacteria this reaction is performed by a single subunit oligosaccharyltransferase on the extended consensus sequence DIE-X1-N-X2-S/T (where neither X1 nor X2 are proline; SEQ ID NO: 35) in the periplasm (for a review see Nothaft H, Szymanski C M: Nat Rev Microbiol. 2010). In addition, it was shown in members of the Pasteurellaceae that the consensus sequence N-X-S/T, wherein X is not proline, can be glycosylated in the cytoplasm (Grass et al., Mol Microbiol. 2003 May; 48(3):737-51; Choi et al., PLoS ONE 5(12): e15888. doi:10.1371/-journal.pone.0015888, 2010; Schwarz et al., 2011, J Biol Chem. 2011 Oct. 7; 286(40):35267-74).

The term Pasteurellaceae protein, a functional fragment or derivative thereof encompasses naturally occurring Pasteurellaceae proteins as well as functional fragments and derivatives thereof having at least one glycosylated N-X-S/T consensus sequence, wherein X is not proline. The term “functional fragment or derivative of a Pasteurellaceae protein” according to the invention is meant to include any naturally occurring Pasteurellaceae protein, fragment or derivative thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc., as long as the fragment or derivative has at least one consensus sequence capable of receiving glycosyl residues, and which will provide for an immune response in a mammal or bird, e.g. antibody production, TH1 and/or TH2 responses. Preferably the functional fragment or derivative of a Pasteurellaceae protein according to the invention has at least 40, preferably at least 50, more preferably at least 70, most preferably at least 80% amino acid sequence identity to a naturally occurring Pasteurellaceae protein.

It is noted that proteins having suitable consensus sequences for N-glycosylation are abundant in Pasteurellaceae. For illustrating their abundance and for describing preferred embodiments of proteins for use in the present invention an exemplifying list of proteins is provided in Table 1 at the end of the experimental section.

Preferably, the Pasteurellaceae protein is a secreted protein, preferably an autotransporter protein, an LPS-assembly protein, a hemagglutinin/hemolysin-like protein, or a RTX-toxin, more preferably a Pasteurellaceae autotransporter adhesin or a Pasteurellaceae LPS-assembly protein.

In a more preferred embodiment the Pasteurellaceae protein is an autotransporter protein, preferably an autotransporter protein of A. pleuropneumoniae or Mannheimia haemolytica, more preferably the Autotransporter adhesin [ataC] A. pleuropneumoniae serotype 7 (strain AP76) (accession no. B3GX20_ACTP7; SEQ ID NO: 1), a functional fragment, homologue or derivative thereof having at least 40, preferably at least 50, more preferably at least 70, most preferably at least 80% amino acid sequence identity to ataC.

It is also preferred that the protein for use as N-glycosylated protein of the invention has more than one, preferably at least 2 to 50, more preferably at least 2 to 30, most preferably at least 5 to 20 consensus sequences.

In an even more preferred embodiment the N-glycosylated protein is selected from the group consisting of:
(1) autotransporter adhesin [ataC] A. pleuropneumoniae serotype 7 (strain AP76) (accession no. B3GX20_ACTP7; SEQ ID NO: 1),
(2) putative uncharacterized protein [APP6_{—}1216] A. pleuropneumoniae serovar 6 strain Femo (accession no. D9P9E6_ACTPL; SEQ ID NO: 2),
(3) autotransporter adhesin [ataB] A. pleuropneumoniae serotype 7 (strain AP76) (accession no. B3GZU4_ACTP7; SEQ ID NO: 3),
(4) autotransporter adhesin [APP2_{—}0255] A. pleuropneumoniae serovar 2 strain 4226 (accession no. D9P3A1_ACTPL; SEQ ID NO: 4),
(5) autotransporter adhesin [APL_{—}0443] A. pleuropneumoniae serotype 5b (strain L20) (accession no. A3MZG1_ACTP2; SEQ ID NO: 5),
(6) putative uncharacterized protein [APP6_{—}2139] A. pleuropneumoniae serovar 6 strain Femo (accession no. D9PD81_ACTPL; SEQ ID NO: 6),
(7) autotransporter adhesin [APL_{—}0104] A. pleuropneumoniae serotype 5b (strain L20; SEQ ID NO: 7),
(8) autotransporter adhesin [appser10_{—}1320] A. pleuropneumoniae serovar 10 strain D13039 (accession no. E0F1W7_ACTPL; SEQ ID NO: 8),
(9) autotransporter adhesin [appser10_{—}5000] A. pleuropneumoniae serovar 10 strain D13039 (accession no. E0F2X9_ACTPL; SEQ ID NO: 9),
(10) autotransporter adhesin [ataA] Actinobacillus pleuropneumoniae serotype 7

(strain AP76) (accession number B3GZU3_ACTP7; SEQ ID NO: 10),
(11) AT family autotransporter/adhesin [MHA_{—}1367] Mannheimia haemolytica PHL213 (accession number A7JTA5_PASHA; SEQ ID NO: 11),
(12) Possible autotransporter/adhesin [MHA_{—}2701] Mannheimia haemolytica PHL213 (accession number A7JWY1_PASHA; SEQ ID NO: 12),
(13) AT family autotransporter/adhesin [COK_{—}1394] Mannheimia haemolytica serotype A2 str. BOVINE (accession number E2P8A5_PASHA; SEQ ID NO: 13),
(14) Autotransporter adhesin [COK_{—}0898] Mannheimia haemolytica serotype A2 str. BOVINE (accession number E2P6W7_PASHA; SEQ ID NO: 14),
(15) Autotransporter adhesin [COK_{—}1916] Mannheimia haemolytica serotype A2 str. BOVINE (accession number E2P9S1_PASHA; SEQ ID NO: 15),
   and homologues thereof in different strains or serotypes of the same species.

The N-linked glycan of the protein of the invention can vary widely and preferably consists of glucose and/or galactose molecules.

Preferably the N-linked glycan is selected from the group consisting of β-Glc/β-Gal, (β-Glc/β-Gal)-1,6-(α-Glc/α-Gal)ₙ, wherein n is at least 1, preferably 1 to 10, preferably 1 to 6, more preferably 2 to 5, more preferably β-Glc-α1,6-Glc-α1,6-Glc, β-Glc-α1,6-Glc-α1,6-Glc-α1,6-Glc, β-Glc-α1,6-Glc-α1,6-Glc-α1,6-Glc-α1,6-Glc, β-Glc-α1,6-Glc-α1,6-Glc-α1,6-Glc-α1,6-Glc-α1,6-Glc, β-Gal-α1,6-Glc-α1,6-Glc, β-Gal-α1,6-Glc-α1,6-Glc-α1,6-Glc, β-Gal-α1,6-Glc-α1,6-Glc-α1,6-Glc-α1,6-Glc and β-Gal-α1,6-Glc-α1,6-Glc-α1,6-Glc-α1,6-Glc-α1,6-Glc.

In a particularly preferred embodiment the N-glycosylated protein is the autotransporteradhesin [ataC] [A. pleuropneumoniae serotype 7 (strain AP76) (accession no. B3GX20_ACTP7; SEQ ID NO: 1), a functional fragment or derivative thereof having at least 40, preferably at least 50, more preferably at least 70, most preferably at least 80% amino acid sequence identity to ataC, wherein at least 10%, preferably at least 30%, more preferably at least 50%, most preferably at least 70% of all N-X-S/T consensus sequences are glycosylated, preferably glucosylated, more preferably glucosylated by β-Glc-α1,6-Glc-α1,6-Glc.

In a most preferred embodiment the N-glycosylated protein according to the invention is
(i) a fragment of autotransporter adhesin [ataC] A. pleuropneumoniae serotype 7 (strain AP76) (accession no. B3GX20_ACTP7; SEQ ID NO: 1), preferably comprising amino acids 1866 to 2516 (SEQ ID NO: 16);
(ii) a fragment of autotransporter adhesin [ataC] A. pleuropneumoniae serotype 7 (strain AP76) (accession no. B3GX20_ACTP7; SEQ ID NO: 1), preferably comprising amino acids 61 to 984 (SEQ ID NO: 17);
(iii) a fragment of autotransporter adhesin [ataC] A. pleuropneumoniae serotype 7 (strain AP76) (accession no. B3GX20_ACTP7; SEQ ID NO: 1), preferably comprising amino acids 51 to 2428 (SEQ ID NO: 18);
(iv) a fragment of autotransporter adhesin [ataC] A. pleuropneumoniae serotype 7 (strain AP76) (accession no. B3GX20_ACTP7; SEQ ID NO: 1), preferably comprising amino acids 1866 to 2428 (SEQ ID NO: 19),
   wherein (i), (ii), (iii) and (iv) are N-glycosylated, preferably glucosylated, more preferably glucosylated by βGlc-α1,6-Glc-α1,6-Glc in
(a) 1 to 14, preferably at least 2 to 10, more preferably 2 to 8 consensus sequence(s) N-X-S/T for fragment (i),
(b) 1 to 9, preferably at least 2 to 8, more preferably 2 to 5 consensus sequence(s) N-X-S/T for fragment (ii),
(c) 1 to 73, preferably at least 2 to 50, more preferably 5 to 20 consensus sequence(s) N-X-S/T for fragment (iii) and
(d) 1 to 13, preferably at least 2 to 10, more preferably 2 to 8 consensus sequence(s) N-X-S/T for fragment (iv).

The N-glycosylated proteins according to the invention are effective in preventing acute disease, preclude colonization to a significant extent and are expected to be widely cross-protective among different serotypes of Pasteurellaceae species due to the common nature of N-glycosylation. Without wishing to be bound by theory, it is assumed that the N-glycosylation of Pasteurellaceae proteins, functional fragments and derivatives thereof improves antigen recognition and has a pronounced effect on Th1 and Th2 helper cells for providing a substantial immune response in related species, thus providing for inter-species cross-protection.

In a further aspect the present invention relates to a pharmaceutical composition comprising a pharmaceutically effective amount of at least one N-glycosylated protein of the invention and optionally one or more pharmaceutically acceptable carriers and/or adjuvants.

Pharmaceutical dosage forms of the N-glycosylated proteins described herein include pharmaceutically acceptable carriers and/or adjuvants known to those of ordinary skill in the art. These carriers and adjuvants include, for example, ion exchangers, alumina, aluminium stearate, lecithin, serum proteins, buffer substances, water, salts, electrolytes, cellulose-based substances, gelatine, water, petrolatum, animal or vegetable oil, mineral or synthetic oil, saline, dextrose or other saccharide and glycol compounds such as ethylene glycol, propylene glycol or polyethylene glycol, antioxidants, lactate, etc. Preferred dosage forms include tablets, capsules, solutions, suspensions, emulsions, reconstitutable powders and transdermal patches. Methods for preparing dosage forms are well known, see, for example, H. C. Ansel and N. G. Popovish, Pharmaceutical Dosage Forms and Drug Delivery Systems, 5^{th} ed., Lea and Febiger (1990) and, in particular, Pastoret et al., Veterinary Vaccinology, Elsevier March 1999). Dosage levels and requirements are well-recognized in the art and may be selected by those of ordinary skill in the art from available methods and techniques suitable for a particular patient. As the skilled artisan will appreciate, lower or higher doses may be required depending on particular factors. For instance, specific doses and treatment regimens will depend on factors such as the patient's (human or animal) general health profile, the severity and course of the patient's disorder or disposition thereto, and the judgment of the treating physician or veterinarian.

In a further aspect, the present invention is directed to a method for producing N-glycosylated proteins of the invention, comprising the following steps:
(i) providing a cell, preferably a prokaryotic cell, more preferably an E. coli cell expressing an N-glycosyltransferase (NGT) and a Pasteurellaceae protein, functional fragment or derivative thereof having at least one N-X-S/T consensus sequence(s), wherein X is not Pro;
(ii) culturing said cell under conditions that lead to the N-linked glycosylation, preferably glucosylation of said Pasteurellaceae protein;
(iii) optionally co-expressing a glycosyltransferase for glycosyl extension of N-linked glycan, preferably for extensions containing glucose residues;
(iv) optionally purifying the N-glycosylated proteins.

The preferred cell for practicing the above-described method of the invention is an E. coli cell, preferably one selected from the group consisting of DH5α, BL21, Top10, W3110, CC118λpir, Sm10λpir, TG1 and XL1Blue (Hanahan 1983; J Mol Biol 166(4): 557-80; Herrero, M., V. de Lorenzo, et al. (1990). J Bacteriol 172(11): 6557-67; Miller, V. L. and J. J. Mekalanos (1988). J Bacteriol 170(6): 2575-83). The expression of the NGT and the Pasteurellaceae protein, functional fragment or derivative thereof can be facilitated with a number of common expression and cloning vectors of different origins of replication like, e.g. pACYC184, pBR322, pET22, pET24, pMLBAD, pBAD, pBluescript and pEC415 (Lefebre, M. D. and M. A. Valvano (2002), Appl Environ Microbiol 68(12): 5956-64; Schulz H, Hennecke H, Thöny-Meyer L. (1998) Science. August 21; 281(5380):1197-200).

The culturing step of the method of the invention is practiced by growth of the bacterial cells under standard laboratory conditions known to those trained in microbiology, e.g. growth of E. coli cells containing plasmids encoding the gene for the NGT as well as the Pasteurellaceae protein containing the consensus sequence in Luria-Bertani medium with the appropriate antibiotics added to select for presence of the compatible expression plasmids. If desired, transcription of the genes of interest from the plasmids can be induced by the addition of a suitable inducer, e.g. isopropyl thiogalactoside (IPTG) or arabinose as commonly used in molecular biology (Schwarz et al., Nat Chem Biol 2010 April; 6(4):264-6; Künzler M et al, Methods Enzymol. 2010; 480:141-50; Lizak C et al., Nature. 2011 Jun. 15; 474(7351):350-5).

For example, for producing an N-glycosylated protein of the invention with further extension of the glycosyl residue, an α6GlcT can be co-expressed in E. coli cells containing plasmids encoding the gene for the NGT, the α6GlcT as well as the Pasteurellaceae protein containing the consensus sequence in Luria-Bertani medium with the appropriate antibiotics added to select for presence of the compatible expression plasmids. If desired, the transcription of the genes of interest from the plasmids can be induced by the addition of a suitable inducer as exemplified above.

Purification of N-glycosylated proteins can be achieved by common methods regularly practiced in biochemistry and molecular biology, e.g. cellular fractionation, precipitation or affinity chromatography. Affinity chromatography is preferred as it yields protein preparations of good quality. Purification of a protein of interest by affinity chromatography can be achieved, e.g. by addition of several histidine residues at the N- or C-terminus of the protein facilitating binding of the protein to a chromatography column containing Ni²⁺ ions or by the interaction of the protein of interest with other positively or negatively charged amino acids on the column material (Schwarz et al., Nat Chem Biol 2010 April; 6(4):264-6; Künzler M et al, Methods Enzymol. 2010; 480:141-50; Lizak C et al., Nature. 2011 Jun. 15; 474(7351):350-5).

The preferred embodiments of the inventive N-glycosylated protein as detailed above with respect to the N-glycoprotein itself, the Pasteurellaceae protein, functional fragments and derivatives thereof as well as the N-linked glycan apply in analogy to the proteins and starting materials produced by the above inventive method. It is additionally noted that it is preferred that the glycosyltransferase used in step (iii) is selected is selected from a Pasteurellaceae protein, preferably proteins of Actinobacillus, Haemophilus, Histophilus and Mannheimia, more preferably proteins from Actinobacillus pleuropneumoniae, Haemophilus influenzae, Haemophilus parasuis, Histophilus somni and Mannheimia haemolytica, most preferably anα6GlcT from A. pleuropneumoniae (Schwarz et al., J Biol Chem. 2011 Oct. 7; 286(40):35267-74).

In a further aspect the present invention relates to a method of diagnosing a bacterial Pasteurellaceae infection comprising the following steps:
(i) providing a sample, preferably selected from blood, saliva, lacrimal, urine and/or feces from a mammal or bird suspected of having a bacterial Pasteurellaceae infection,
(ii) contacting said sample, a component or derivative thereof with an N-glycosylated protein of the invention under conditions that allow for antibody binding, and
(iii) determining binding of said sample, a functional component or derivative thereof to an N-glycosylated protein of the invention.

The term “sample, a functional component or derivative thereof” in the above context means that the sample, e.g. blood, can be partially purified and/or derivatized. For example, the serum fraction of blood can be used as a functional component of the blood sample. Any fraction, component or derivative of the sample can be used for practicing the diagnostic method of the invention that still contains antibodies originally present in the sample.

The N-glycosylated proteins of the present invention can be used as a diagnostic tool to detect antibodies prevailing in mammals and birds, preferably in livestock after Pasteurellaceae infections. These antibodies can bind to the N-glycosylated proteins of the invention and this binding can be detected by suitable and common methods. For example, detection is possible by coupling the N-glycosylated protein(s) of the invention to plates made of non-reactive polymers like polystyrene in a 96- or 394-well format with the methods known to molecular biologists. After incubation of the N-glycosylated protein(s) of the invention with an animal's antibodies those not bound to the N-glycosylated protein can be washed off, whereas those bound to the N-glycosylated protein(s) of the invention can be detected with appropriate secondary antibodies to determine isotypes of antibodies prevailing during and after infection with Pasteurellaceae (Cawthraw S et al., 1994 Avian Diseases 38:341-349).

In another aspect, the present invention relates to a method of treating and/or protecting mammals or birds having or being prone to develop a bacterial Pasteurellaceae infection comprising the administration of a therapeutically effective amount of at least one N-glycoprotein, functional fragment or derivative thereof or a pharmaceutical composition of the invention to a patient, bird or mammal, in need thereof.

In a preferred embodiment the N-glycosylated protein to be administered is a Pasteurellaceae protein, preferably a protein from the genera Actinobacillus, Aggregatibacter, Avibacterium, Basfia, Bibersteinia, Chelonobacter, Gallibacterium, Haemophilus, Histophilus, Lonepinella, Mannheimia, Nicoletella, Pasteurella, Phocoenobacter and Volucribater, more preferably a protein of Actinobacillus, Haemophilus, Histophilus and Mannheimia, most preferably a protein from Actinobacillus pleuropneumoniae, Haemophilus influenzae, Haemophilus parasuis, Histophilus somni and Mannheimia haemolytica.

For therapeutic and/or prophylactic use the pharmaceutical compositions of the invention may be administered in any conventional dosage form in any conventional manner. Routes of administration include, but are not limited to, intravenously, intramuscularly, subcutaneously, intranasally, intrasynovially, by infusion, sublingually, transdermally, orally (e.g. tablet, gavage), topically or by inhalation. The preferred modes of administration are oral, intramuscular, subcutaneous, intravenous and intranasal, intramuscular and subcutaneous being most preferred.

The N-glycosylated proteins of the invention may be administered alone or in combination with adjuvants that enhance stability and/or immunogenicity of the medically effective compounds, facilitate administration of pharmaceutical compositions containing them, provide increased dissolution or dispersion, increase propagative activity—if cells are involved, e.g. cells producing the medically effective compounds, provide adjunct therapy, and the like, including other active ingredients.

The following tables, figures and examples are merely illustrative of the present invention and should not be construed to limit the scope of the invention as indicated by the appended claims in any way.

### BRIEF DESCRIPTION OF THE FIGURES

FIG. 1 Electrophoretic analysis demonstrating the modification of tamra-labeled peptides by putative glycosyltransferases. The reaction products were separated by Tricine-SDS-PAGE analysis and fluorescent signals were acquired by an image analyzer.

FIG. 2 Tricine-SDS-PAGE analysis of glycosylation products. Tamra-labeled peptides were incubated with NGT and different UDP-monosaccharide donors in the presence or absence of EDTA.

FIG. 3 MALDI-Mass spectrometry analysis of tamra-peptide (upper panel) in presence of NGT and UDP-glucose (middle) or UDP-galactose (low).

FIG. 4 Tricine-SDS-PAGE of glucosylated tamra-labeled peptides incubated with α6GlcT and different UDP-monosaccharide donors.

FIG. 5 MALDI-MS analysis of glucosylated products shows that the glucosyltransferase α6GlcT elongates the N-linked glucose with up to six units of glucose in presence of a 1:1000 acceptor:donor ratio.

FIG. 6 Immunoblot of whole cell extracts of E. coli expressing truncated Atac (cAtaC) in the presence (lane 1) and absence (lane 2) of NGT. Note the mobility shift and stabilization of cAtaC upon co-expression with NGT (lane 1).

FIG. 7 Immunoblot of whole cell extracts of E. coli expressing truncated Atac (cAtaC) in the presence (lane 1) and absence (lane 2) of NGT. The sera used for detection originate from an A. pleuropneumoniae-negative pig (left) or a pig infected with A. pleuropneumoniae serotype 9. Note the glycosylation-specific reaction of the APP-positive serum.

FIG. 8 Immunoblot of whole cell extracts of E. coli expressing different substrate proteins for glycosylation in the presence and absence of NGT. The left panel of all parts of the figure shows the detection with an anti-His4-antibody for the presence of protein; the right panel shows the detection with the anti-N-glucose serum. A: Truncated Mannheimia haemolytica COK1394, B: truncated M. haemolytica COI1702, C: short truncated form of A. pleuropneumoniae AtaC, termed scAtaC. D: Actinobacillus toxins ApxI and ApxII.

FIG. 9 ELISA analysis of pigs immunised with glycosylated or unglycosylated scAtaC or adjuvant. Note the reaction of the glycosylation-specific reaction of the animal immunised with glycosylated scAtaC.

### EXAMPLES

### Materials and Methods

Restriction enzymes were purchased from Fermentas. T4 DNA ligase was from NEB. UDP-Glc, UDP-GlcNAc, and UDP-GalNAc were from Sigma. UDP-Gal was obtained from VWR International. Synthetic peptides were purchased from JPT Peptide Technologies.

### Construction of Plasmids

E. coli DH5α was chosen as host for cloning. The ngt ORFs were amplified by PCR using genomic DNA from Y. enterocolitica strain 8081, A. pleuropneumoniae strain L20 or A. pleuropneumoniae strain AP76 as templates. Fragments containing the ngt gene were cut with XhoI and ligated into pEC(AcrA-cyt), previously digested with NdeI, blunted by treatment with Klenow fragment, and digested with XhoI. All ORFs were in frame with a hexa-histidine tag at the C-terminus. All plasmid constructs were verified by sequencing of relevant fragments (Microsynth AG).

### Protein Expression, Purification, and Analysis

E. coli DH5α cells harboring a plasmid for expression of a relevant protein were grown in volumes of 1 l at 37° C. in LB medium. Ampicillin (100 mg/l) or chloramphenicol (25 mg/l) was added to the medium as needed. When cultures reached 0.5 OD/ml, 0.2% arabinose or 1 mM IPTG was added for induction of protein expression. Cells were harvested by centrifugation, resuspended in 30 mMTris pH 8 300 mMNaCl supplemented with 1 mM EDTA and 1 g/l lysozyme, and incubated for one hour at 4° C. MgCl₂ and DNase I (Roche) were added to a final concentration of 5 mM and 0.1 mg/ml, respectively. Cells were broken by French press. Extracts were spun for 30 minutes at 150,000 g at 4° C. The supernatant was supplemented with 20 mM imidazole and loaded on a HisTrap column (GE Healthcare). Purification was done according to the indications given by the provider. Purification of XcOGT was performed according to a published procedure (Clarke et al. (2008) Embo J 27, 2780-2788). Buffer exchange to 25 mM Tris pH 7.2 150 mM NaCl was performed by gel filtration chromatography using HiTrap desalting columns (GE Healthcare). Proteins were analyzed by SDS-PAGE and quantified by measuring absorbance at 280 nm.

### Glycosylation Analysis of Synthetic Peptides

Enzymatic activity using different sugar donors or peptide acceptors was assessed using 1.4 μg (0.46 μM) of NGT and/or α6GlcT in a 50 μl final volume of Tris buffer (pH 7.2, 25 mM). Acceptor peptides and sugar donors were mixed at a 1:100 molar ratio. Glycosylation reactions were run for 16 hours at 30° C. For removal of salts and enzyme, peptides were bound to a C18 cartridge (Sep-Pak Cartridge, Waters) or to a C18 zip-tip (Millipore), washed with 0.1% formic acid, and eluted with a solution of 70% acetonitrile 0.1% formic acid. Analysis of the reaction products was performed by either MALDI-TOF/TOF mass spectrometry, NMR, or gel electrophoresis. For electrophoretic analysis, tamra-labeled peptides were supplemented with reducing sample buffer (0.0625 M Tris-HCl, pH 6.8, 2% SDS (v/w), 5% 3-mercaptoethanol (v/v), 10% glycerol (v/v), 0.01% bromophenol blue (w/v)), boiled at 95° C. for 5 minutes, and separated by Tricine-SDS-PAGE (11). Fluorescence was acquired by an RX Imager (BioRad).

### Glycosylation Analysis of Proteins

50 μg of AcrA were incubated with 1 mM UDP-Glc and 1.4 μg NGT in 25 mM Tris pH 7.2 150 mM NaCl, for 16 hours at 30° C. Samples were digested with trypsin (Promega) overnight at 37° C. Peptides were bound to a C18 cartridge for removal of salts, eluted with a solution of 70% acetonitrile 0.1% formic acid and subjected to MS analysis.

### Example 1

### A. pleuropneumoniae and Yersinia enterocolitica HMW1C Homologues Modify a DANYTK Peptide

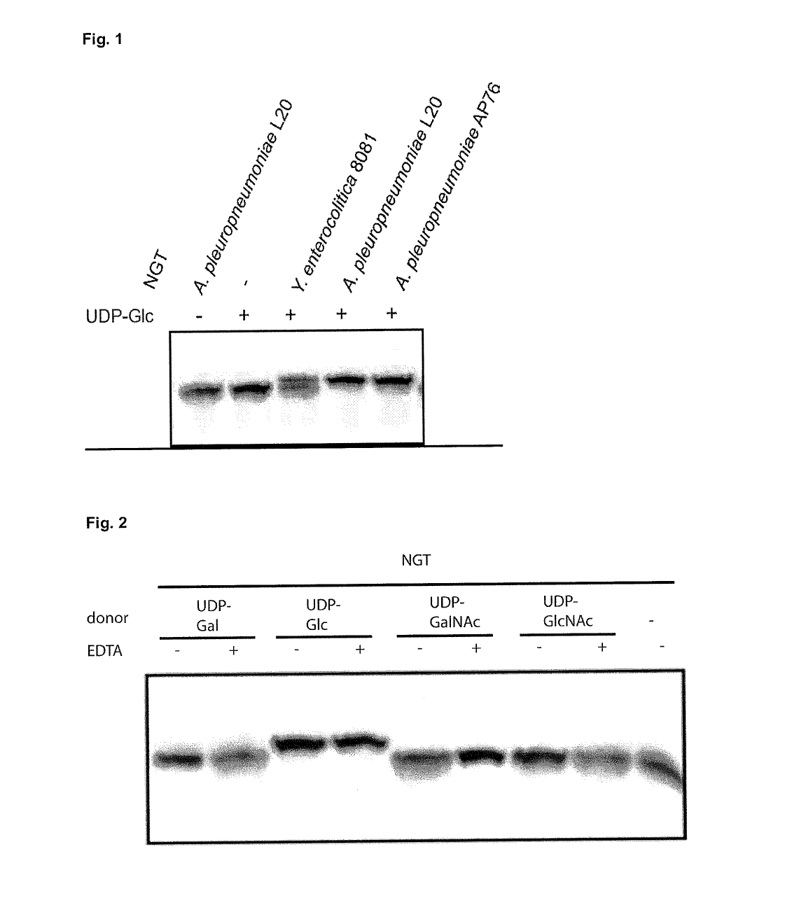

HMW1C homologs from Y. enterocolitica strain 8081, A. pleuropneumoniae strain L20, A. pleuropneumoniae strain AP76, and X. campestris ATCC 33913 were expressed in Escherichia coli and purified. In order to test the proteins for glycosyltransferase activity an in vitro assay developed for analysis of OST activity (Kohda et al. (2007) Glycobiology 17, 1175-1182) was adapted. The purified proteins were incubated with UDP-Glc and a hexapeptide DANYTK (SEQ ID NO: 20) labeled at the N-terminus with a fluorescent dye, carboxytetramethylrhodamine (tamra). After separation of the reaction products by Tricine-SDS-PAGE and detection of fluorescence signals, it was observed that Y. enterocolitica and the two A. pleuropneumoniae homologs modified the tamra-labeled peptide, visualized by a shift in electrophoretic mobility (FIG. 1). By contrast, XcOGT did not exhibit glycosyltransferase activity for this acceptor peptide in the presence of UDP-Glc, UDP-Gal, UDP-GlcNAc, or UDP-GalNAc (data not shown). In the following, the focus is on the A. pleuropneumoniae strain AP76 enzyme.

A. pleuropneumoniae HMW1C Homolog Accepts Glc and Gal as Substrates

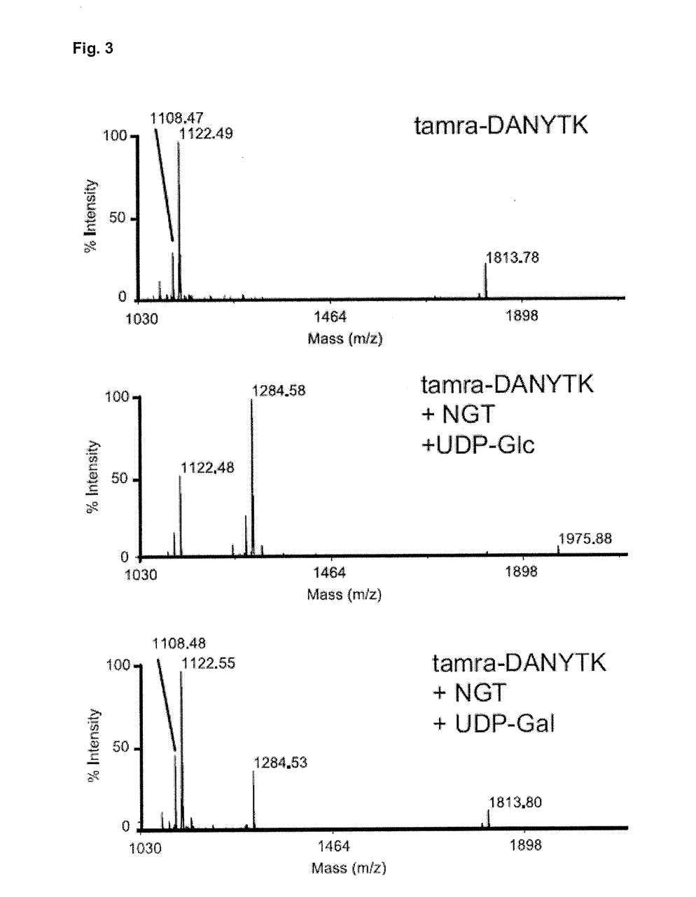

Next, the donor specificity of this NGT in vitro was analyzed. The enzyme transferred glucose or galactose, but not GlcNAc nor GalNAc, to the DANYTK (SEQ ID NO: 20) peptide (FIG. 2). In presence of a 100-fold molar ratio of donor:acceptor, the conversion to glycopeptide was quantitative in the presence of UDP-Glc, while it was marginal in the presence of UDP-Gal. Importantly, NGT glycosylated the peptide in presence of EDTA, proving that glycosyl transfer did not require metal ions. Also, the products of the reaction were monitored by mass spectrometry (FIG. 3). Analysis of unmodified tamra-DANYTK resulted in two major species matching with tamra-DANYTK (calculated MW: 1122.19 Da, observed: 1122.49 Da), and a by-product tamra-(DANYTK)₂ (calculated MW: 1814.50 Da, observed MW: 1813.78 Da). After incubation with NGT and UDP-Glc species corresponding to tamra-DANYTK-Glc (calculated MW: 1284.35 Da, observed MW: 1284.58 Da) and tamra-(DANYTK)₂-Glc (calculated MW: 1975.66 Da, observed MW: 1975.88 Da) were detected. A similar result was obtained after incubation with UDP-Gal. In all cases, addition of a single hexose moiety to the asparagine residue was observed.

A. pleuropneumoniae Encodes for a Processive Glucosyltransferase that Elongates N-Linked Glucose

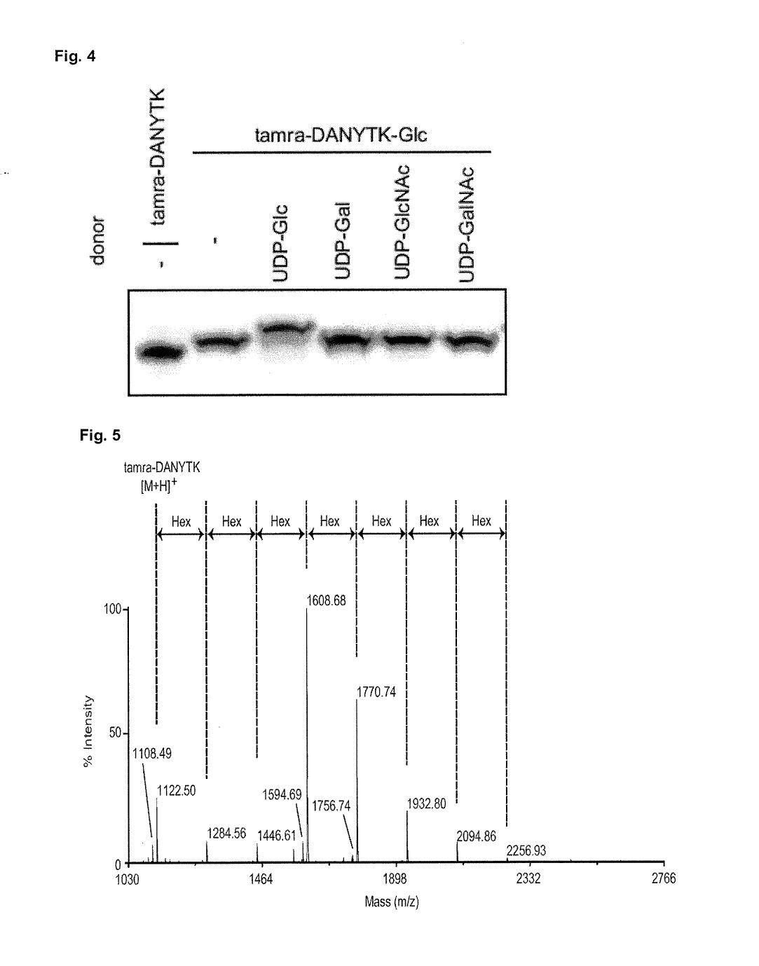

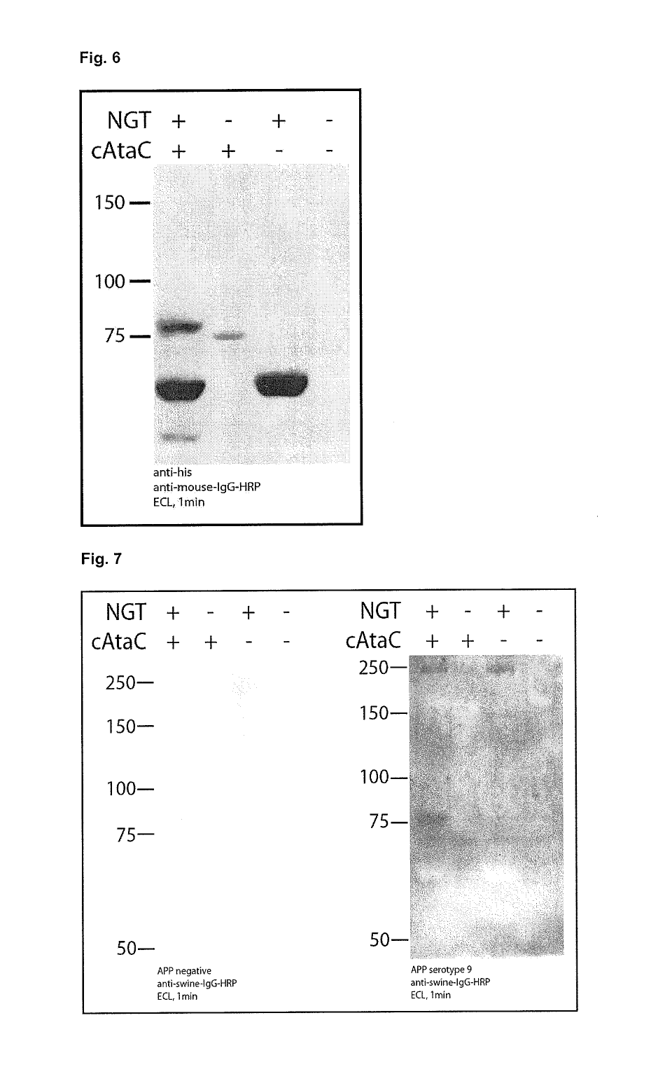

The NGT-encoding genomic region of A. pleuropneumoniae strain AP76 was investigated. This region contains genes encoding for putative proteins involved in the uptake of mannitol and its conversion to glucosamine-6-phosphate, two isomerases, a nucleosidase, and a methylthiotransferase. Interestingly, the ORF next to ngt encodes for a putative glycosyltransferase (APP7_{—}1696). A C-terminally tagged protein was expressed in E. coli. When incubating the purified protein with the tamra-labeled product of the NGT reaction a mobility shift upon tricine-SDS-PAGE indicative of elaboration of the glycopeptide was detected (FIG. 4). This modification occurred in the presence of UDP-Glc, but not in the presence of UDP-Gal, UDP-GlcNAc, or UDP-GalNAc. The glucosyltransferase activity appeared to be cation-independent. The reaction product was analyzed by MS and an addition of two glucose moieties in the presence of a 1:100 acceptor:donor ratio was found. Notably, addition of up to six glucose units was observed in the presence of excess of the donor and with increasing amounts of glucosyltransferase (FIG. 5). MS/MS analysis of the precursor ions corresponding to glucosylated peptides (m/z=1446.61, 1608.68, 1770.74, 1932.80, 2094.86, and 2256.93) gave fragmentation compatible to modified tamra-DANYTK (SEQ ID NO: 20) (data not shown).

In order to determine the chemical structure of the reaction product, the glycopeptide was analyzed by NMR spectroscopy. The ¹H-¹³C HSQC spectrum displayed signals of three different glucose units (data not shown). Two new signals appeared in the anomeric region at ˜100 ppm, in addition to the previously observed signal at ˜82 ppm originating from the Asn-linked glucose. The signals were assigned with a 2D TOCSY and ¹H-¹³C long-range correlations via J couplings. The first set of signals belonging to the N-linked glucose displayed a C6 chemical shift of 68.3 ppm that differed from the initial glycopeptide harboring only a single glucose unit (C6: 63.3 ppm). This was indicative of a carbohydrate attachment at O6. The signals of a second glucose unit originated from a terminal glucose (C6: 63.2 ppm), whose chemical shifts coincided to those of Glc-α1,6-Glc. The third set of signals displayed chemical shifts of a bridging glucose unit that is α1,6-linked on either side. Chemical shifts of Glc-α1,6-Glc-α1,6-Glc reported previously (Hansen et al. (2008) Biopolymers 89, 1179-1193) fitted perfectly the experimental data of the terminal and bridging glucose, providing strong evidence for two α1,6-linked glucose residues. Chemical shifts calculated with the algorithm CASPER (26) further supported the assignment. Therefore, APP7_{—}1697 gene encodes for a processive α1,6 glucosyltransferase (α6GlcT) that elongates the product of the NGT reaction.

### NGT Exhibits Acceptor Site Specificity Overlapping to OST

Glycosylation of several peptides representing model glycoproteins from yeast containing the N-X-S/T consensus sequence in different positions (N-terminus, central, C-terminus) was examined. Among these were the following: Amino acids (AA) 100-120 of the yeast glycoprotein PRY3, AA 268-288 of GAS, AA 102-117 of EXG2, AA 212-226 of PLB2, AA 412-434 of PDI1 and AA 352-374 of APE3. Glycosylation at N-X-S/T site was observed for all peptides (data not shown). Although our analysis by MS/MS was not quantitative, the position of the consensus sequence within the peptide seemed not to affect glycosylation and a sequence preference for S or T at the +2 position was not observed. Moreover, it appeared that the N-X-S/T sequence was the minimal primary acceptor consensus recognized by NGT. Importantly, glycosylation of short peptides such as DQNAT (SEQ ID NO: 21) or DFNVT (SEQ ID NO: 22) (data not shown), known substrates identified in vitro for the bacterial OST from C. jejuni (Chen, M. M., Glover, K. J., and Imperiali, B. (2007) Biochemistry 46, 5579-5585) was not detected.

In summary, the above-described experimental investigations leading to this invention demonstrate that N-glycosylated proteins can be produced from essentially any consensus site-containing proteins, which are found abundantly throughout nature, in particular in bacteria.

### Example 2

### Actinobacillus pleuropneumoniae and Mannheimia haemolytica NGT Glycosylate Heterologous Proteins

To further investigate the protein substrate specificity of Pasteurellaceae NGTs, the NGT from A. pleuropneumoniae or M. haemolytica was expressed in E. coli cells in the presence of different substrate proteins. The proteins used for illustrative purposes were truncated forms of the M. haemolytica proteins COK_{—}1394 and COI_{—}1702 as well as a further truncated form of cAtaC termed scAtaC. The cloning of these constructs is described in table 2 where also the sources of the original cloning vectors and expression plasmids are listed. In more detail, BL21 (DE3) cells were transformed with one of the following plasmid combinations
a) pMLBAD+pET24b-COK_{—}1394-HIS10
b) pMLBAD-Mh.NGTmyc+pET24b-COK_{—}1394-HIS10
c) pMLBAD-AP1697myc+pET24b-COK_{—}1394-HIS10
d) pMLBAD+pET24b-COI_{—}1702-HIS10
e) pMLBAD-AP1697myc+pET24b-COI_{—}1702-HIS10
f) pMLBAD-Mh.NGTmyc+pET24b-COI_{—}1702-HIS10
g) pMLBAD-AP1697myc+pET24b-ApxI
h) pMLBAD-AP1697myc+pET24b-ApxII
i) pMLBAD-AP1697myc+pET24b (control)
j) pMLBAD+pET24b (control)
   for testing expression and glycosylation of the proteins encoded by these plasmids. Cells were inoculated from an overnight culture to an OD₆₀₀/ml of 0.05. After incubation at 37° C. shaking to an OD₆₀₀/ml=0.3-0.5 expression of the NGT was induced by the addition of arabinose to a final concentration of 0.2% and the cell cultures were transferred back to 37° C. 2 h later IPTG was added to a final concentration of 0.5 mM. After further growth at 37° C. for 4 h cells were spun down and prepared for analysis by SDS-PAGE followed by immunoblot: 1 OD₆₀₀ per cell line was harvested by centrifugation at 10.000 g for 2 min at room temperature. The supernatant was discarded and the pellet resuspended in 100 μl 1× Lämmli sample buffer and incubated 10 min at 95° C. The immunoblot was performed with the monoclonal mouse anti-His4-antibody (Qiagen) to detect the substrate proteins as well as with the human glycan-specific serum SR168 (courtesy of AM Papini, Florence). The detection of the bound antibodies was performed with an anti-mouse-IgG-HRP conjugate for the first and an anti-human-IgG-HRP conjugate for the latter primary antibody. The bound conjugate was visualised by incubation with ECL (GE Healthcare).

To obtain high amounts of glycosylated HIS10-scAtaC, this protein was co-expressed in DH5α cells on a pMLBAD-plasmid in combination with the A. pleuropneumoniae NGT in pEC415 or as a control pEC415 not containing the NGT. Similar to the strains for COK_{—}1394 and COI_{—}1702-expression mentioned above, induction of scAtaC with arabinose to a final concentration of 0.2% was at an OD600/ml=0.3-0.5. The cells were grown for 6 h post induction before harvesting. Detection of the protein by SDS-PAGE and immunoblot was performed as described above for the other proteins.

As demonstrated in FIG. 8, all proteins were detected by the anti-His₄-immunoblot, thus demonstrating the presence of the proteins after induction (left panels of FIG. 8A, B, C). In the presence of the NGT a shift to a higher molecular weight was observed with the anti-N-glucose specific serum SR168 (right panels of FIG. 8A, B, C). This was the result of the addition of glucose. Consequently, the experiment proved that also in vivo a Pasteurellaceae NGT has broad substrate specificity with regard to the protein substrate and that it can be utilized to glycosylate many different proteins.

### Example 3

The immunogenicity of the glycosylated proteins COK1394 and scAtaC as well as unglycosylated scAtaC as control was tested in piglets. The proteins were purified via Ni-NTA-agarose with the help of an N-terminal His₁₀-tag.

Briefly, cell pellets of induced cells were resuspended in 40 ml 30 mM TrisHCl, pH 8, 300 mM NaCl, 1× protease inhibitor cocktail complete EDTA-free (Roche, catalogue number 11873580001) 0.1 mg/ml DNaseI (Fermentas) and cells were broken using a French Press. Two centrifugation steps (10 min at 4° C. and 3860 g followed by 30 min at 15.000 g at 4° C.) yielded the soluble fraction of the cells. This was loaded onto HisTrap HP 1 ml column which was equilibrated in 30 mM TrisHCl pH 8, 300 mM NaCl. The column was washed on an Aekta FPLC-System (Amersham Biosciences) with 17 ml 30 mM TrisHCl, pH 8, 300 mM NaCl, 30 mM imidazole before the bound target protein was eluted in 1 ml fractions with 200 mM imidazole in 30 mM TrisHCl, pH 8, 200 mM NaCl. The elution fractions were pooled and dialysed (Spectra/Por 25K MWCO; Spectrum Labs) overnight against 30 mM TrisHCl, pH 8, 300 mM NaCl at 4° C. For the glycosylated proteins a further purification step under denaturing conditions was included to separate the glycosylated protein from the NGT. For this purpose the dialysate of the glycosylated protein was adjusted to 6 M urea+10 mM DTT and incubated 60 min at 60° C. during which the DTT was added again to a 10 mM final concentration after 30 min. All following steps were performed at RT. The sample was added on top of a self-packed Ni-NTA Agarose column of 1 ml, equilibrated with 6 M urea, 10 mM DTT in 30 mM TrisHCl, pH 8, 300 mM NaCl. After sample load, the column was washed with 20 ml 6 M Urea, 10 mM DTT in 30 mM TrisHCl, pH 8, 300 mM NaCl followed by a washing step with 20 ml 6 M urea, 10 mM DTT, 30 mM imidazole in 30 mM TrisHCl, pH 8, 300 mM NaCl followed by another washing step with 20 ml 6 M urea, 10 mM DTT, 40 mM imidazole in 30 mM TrisHCl, pH 8, 300 mM NaCl. The bound protein was eluted in 1 ml fractions with 6 M urea, 10 mM DTT, 100 mM imidazole in 30 mM TrisHCl pH 8, 300 mM NaCl. The elution fractions were pooled and dialysed (Spectra/Por 25K MWCO; Spectrum Labs) over night against 30 mM TrisHCl, pH8, 300 mM NaCl at 4° C. For the animal experiments the protein concentration was determined by BCA-assay and the proteins were aliquoted to 800 μg per tube for lyophilisation overnight. For injection into the animals the lyophilized proteins were re-suspended in 2 ml Diluvac forte (Merck).

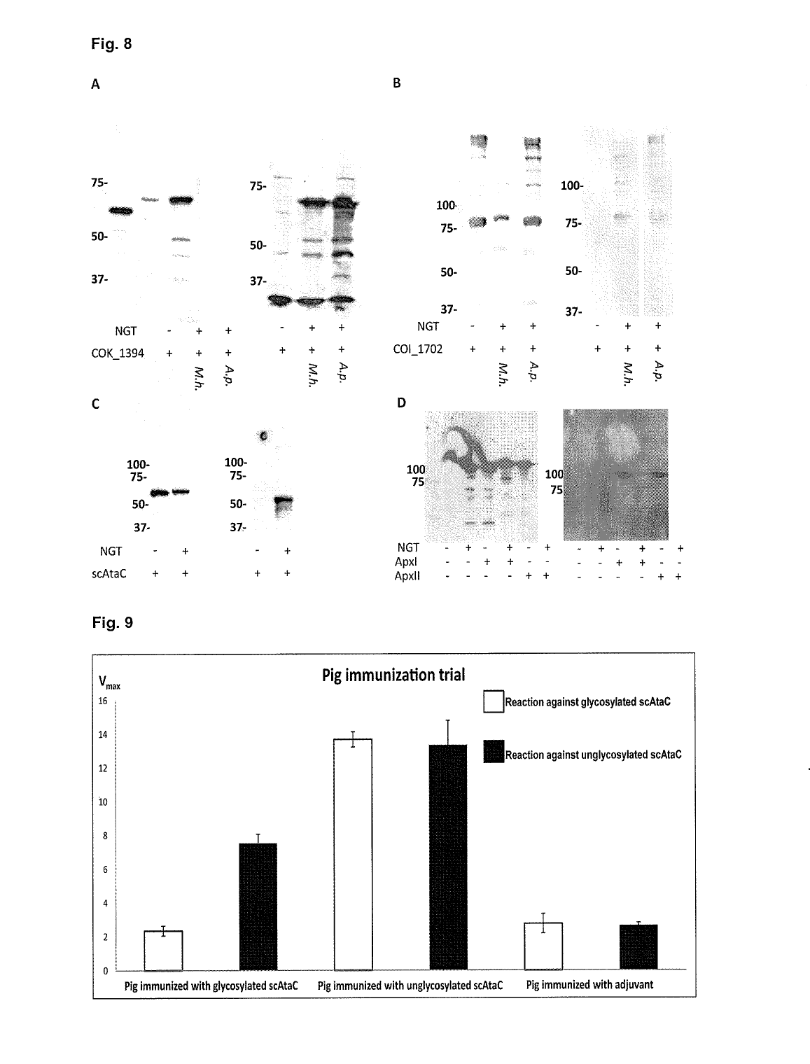

Animal experiments were approved by the Swiss authorities (Kantonales Veterinäramt, Zurich, Switzerland, license number 177/2011) and performed according to the legal requirements. The 8-weeks-old piglets were allowed food and water ad libitum. 18 animals were randomly distributed into different groups: 4 animals received adjuvant (Diluvac Forte, Merck), 4 animals received glycosylated scAtaC (800 μg/pig), 4 animals unglycosylated scAtaC (800 μg/pig) and 6 animals glycosylated COK1394 (800 μg/pig) intramuscularly. None of the animals had an adverse or allergic reaction to the injection. 4 weeks post primary injection blood was taken and the serum prepared. Serum was tested by ELISA for antibodies against the injected proteins. The ELISA was carried out as follows: Plates were coated over night at 4° C. with glycosylated and unglycosylated scAtaC (both with N-terminal His₁₀-tag), 500 ng protein per well. The next day, protein in coating solution was discarded. After washing once with PBS+0.05% Tween-20 the plates were blocked with 5% milk in PBS+0.05% Tween-20 for 1 h at room temperature. After discarding the blocking solution the plate was incubated with the sera diluted 1:400 in 5% milk in PBS+0.05% Tween-20 for 1 h at RT. The sera were distributed on the plate in such a way that each serum was tested against glycosylated and unglycosylated scAtaC in duplicates. After this 1 h incubation the sera were discarded and the plates were washed five times with PBS+0.05% Tween-20. To detect the bound antibodies in the sera the plates were then incubated with an anti-swine-IgG-HRP conjugate (Santa Cruz #sc-2463), diluted 1:1000 in 5% milk in PBS+0.05% Tween-20 for 1 h at room temperature. After this incubation with the conjugate solutions were discarded and the plates were washed five times with PBS+0.05% Tween-20. To visualize the bound conjugate the plates were developed with ABTS (2,2′-azino-bis(3-ethylbenzthiazoline-6-sulphonic acid; final concentration 1 mM in 70 mM phosphocitrate buffer pH 4.2 with addition of 0.03% H₂O₂) by measuring the absorption at 405 nm every 20 s for 200 s leading to the maximal velocity (Vₘₐₓ) of the color development. The average and standard deviation of this value for each serum on glycosylated and unglycosylated scAtaC were then calculated and plotted. Examplary data of 3 piglets are shown in FIG. 9. The serum of the piglet injected with glycosylated scAtaC gave a good reaction against glycosylated scAtaC and less signal for the unglycosylated scAtaC. Therefore, the injection of glycosylated scAtaC leads to an immune response including anti-glycan antibodies. When a piglet was injected with unglycosylated scAtaC the signal obtained from glycosylated and unglycosylated scAtaC was approximately equal. This demonstrates that the antibodies produced are directed against the protein part of scAtaC. If the serum of an animal injected with adjuvant was tested, only a background reaction against glycosylated and unglycosylated scAtaC was detectable. There was no immune response against these proteins in this animal.

TABLE 1
Examples of Pasteurellaceae proteins with suitable glycosylation
consensus sequences as identified by sequence analysis

| | | Number of glycosylation |
|---|---|---|
| Organism | Protein(s) | consensus sequences |
| Actinobacillus pleuropneumoniae | Autotransporter adhesins | Between 3-92, |
| | ataA*, ataB*, ataC*; LPS- | depending |
| | assembly protein IptD*; | on protein |
| | Hemagglutinin/hemolysin-like | |
| | protein*; RTX-II toxin | |
| | determinant A*; RTX-I toxin | |
| | determinant A*, RTX-III toxin | |
| | determinant A*; RTX-IV toxin | |
| | determinant A* | |
| Aggregatibacter actinomycetemcomitans | Leukotoxin; | Between 6-46, |
| | autotransporter/adhesin*; | depending |
| | | on protein |
| Bibersteinia trehalosi | Leukotoxin | 5 |
| Gallibacterium anatis | Putative | Between 5-28, |
| | autotransporter/adhesin*; | depending |
| | RTX toxin GTX* | on protein |
| Haemophilus influenzae | Adhesion and penetration | 27 |
| | protein autotransporter | |
| Histophilus somni | YadA domain protein* | Between 25-66, |
| | | depending |
| | | on protein |
| Mannheimia haemolytica | Autotransporter adhesin*; | Between 9-40, |
| | LPS-assembly protein IptD* | depending |
| | | on protein |
| Pasteurella aerogenes | Exotoxin paxA | 8 |

*Homologs in same and different strains (e.g. of different serotypes) of the same organism included

TABLE 2
List of oligonucleotides and plamids used in this studyOligonucleotides

| Name | Sequence | Usage |
|---|---|---|
| COK_1394 for | GAACCATGGGGGAT | Amplification of COK_1394 from genomic |
| | AAGTCTGTTGCAAAT | DNA of wild type Mannheimia haemolytica |
| | (SEQ ID NO: 23) | isolates (courtesy of Tierspital, Uni Zürich) |
| | | for cloning into pET24b+ with restriction |
| | | enzymes NcoI and XhoI |
| COI_1315 rev | CTTCTCGAGTTAGTG | Amplification of COK_1394 from genomic |
| | ATGATGATGATGATG | DNA of wild type Mannheimia haemolytica |
| | GTGGTGGTGGTGAT | isolates (courtesy of Tierspital, Uni Zürich) |
| | CCGGATTCACAACAG | for cloning into pET24b+ with restriction |
| | AACCG (SEQ ID NO: | enzymes NcoI and XhoI |
| | 24) | |
| COI_1702 for | GAACCATGGTAATCA | Amplification of COI_1702 from genomic |
| | AGGCTAACACTACGG | DNA of wild type Mannheimia haemolytica |
| | CATTAAATGAT (SEQ | isolates (courtesy of Tierspital, Uni Zürich) |
| | ID NO: 25) | for cloning into pET24b+ with restriction |
| | | enzymes NcoI and XhoI |
| COI_1702 rev | GTTCTCGAGTTAATG | Amplification of COI_1702 from genomic |
| | ATGATGATGATGGTG | DNA of wild type Mannheimia haemolytica |
| | ATGATGGTGGTGCTG | isolates (courtesy of Tierspital, Uni Zürich) |
| | CGGTAACGCTGCTG | for cloning into pET24b+ with restriction |
| | CATTT (SEQ ID NO: | enzymes NcoI and XhoI |
| | 26) | |
| NGT M.h. for | CAGAATTCATGTCAG | Amplification of N-glycosyltransferase |
| | CAGAAAATATGCCTA | from genomic DNA of wild type |
| | G (SEQ ID NO: 27) | Mannheimia haemolytica isolates |
| | | (courtesy of Tierspital, Uni Zürich) for |
| | | cloning into pMLBAD with restriction |
| | | enzymes PstI and EcoRI |
| NGT M.h. rev | GTTCTGCAGCTACAG | Amplification of N-glycosyltransferase |
| | ATCCTCTTCTGAGAT | from genomic DNA of wild type |
| | GAGTTTTTGTTCGCT | Mannheimia haemolytica isolates |
| | CTTAGTTTCGGTTTTT | (courtesy of Tierspital, Uni Zürich) for |
| | GC (SEQ ID NO: 28) | cloning into into pMLBAD with restriction |
| | | enzymes PstI and EcoRI |
| APP76_1967myc 5′ | GAACATATGGAAAAC | Amplification of A. pleuropneumoniae N- |
| | GAAAATAAACCGAAT | glycosyltransferase from pFLA91 |
| | GTA (SEQ ID NO: 29) | (Schwarz et al., J Biol Chem. 2011 Oct. |
| | | 7; 286(40):35267-74) for cloning into |
| | | pEC415 with restriction enzymes NdeI |
| | | and EcoRI |
| APP76_1967myc 3′ | CGAATTCTACAGATC | Amplification of A. pleuropneumoniae N- |
| | CTCTTCTGAGATGAG | glycosyltransferase from pFLA91 |
| | TTTTTGTTCGTCGAC | (Schwarz et al., J Biol Chem. 2011 Oct. |
| | CTCGAGATTTTCTTTT | 7; 286(40):35267-74) for cloning into |
| | AGGAACG (SEQ ID | pEC415 with restriction enzymes NdeI |
| | NO: 30) | and EcoRI |
| AP76NGT1 | CATCCATGGAAAACG | Amplification of A. pleuropneumoniae |
| | AAAATAAACCGAAT | NGT from pEC415-AP1697myc (s. below) |
| | (SEQ ID NO: 31) | for cloning into pMLBAD with restriction |
| | | enzymes NcoI and PstI |
| AP76NGT2 | CACTGCAGAATTCTA | Amplification of A. pleuropneumoniae |
| | CAGATCCTCTTCTGA | NGT from pEC415-AP1697myc (s. below) |
| | (SEQ ID NO: 32) | for cloning into pMLBAD with NcoI and |
| | | PstI |
| APL0443_T2_FP | AAACCATGGCAACCC | Amplification of A. pleuropneumoniae |
| | TTAAAGATGGCTTAA | AtaC from genomic DNA (courtesy of |
| | AATTC (SEQ ID NO: | Universität Bern) for cloning into pBAD |
| | 33) | with NcoI and KpnI |
| APL0443_T2_RP | TTTGGTACCTTATAC | Amplification of A. pleuropneumoniae |
| | CATTGATAACCTACA | AtaC from genomic DNA (courtesy of |
| | CCTAC (SEQ ID NO: | Universität Bern) for cloning into pBAD |
| | 34) | with NcoI and KpnI |

Plasmids

| Name | Phenotype and promoter type | Source |
|---|---|---|
| pET24b(+) | Kanamycin resistant, T7 promoter | Novagen |
| pMLBAD | Trimethoprim resistant, Pₐᵣₐ | Lefebre and Valvano, |
| | | Appl Environ Microbiol. |
| | | 2002 December; 68(12):5956- |
| | | 64 |
| pBAD/Myc-His | Ampicillin resistant, Pₐᵣₐ | Invitrogen |
| pEC415 | Ampicillin resistant, Pₐᵣₐ | Enggist et al., J |
| | | Bacteriol. 2003 |
| | | January; 185(1): 175- |
| | | 183 |
| pET24b- | Kanamycin resistant, expression of M. | This study |
| COK_1394_HIS10 | haemolytica COK_1394 with C-terminal | |
| | His₁₀ under control of T7-prmoter | |
| pET24b-COI_1702- | Kanamycin resistant, expression of M. | This study |
| HIS10 | haemolytica COI_1702 with C-terminal | |
| | His₁₀ under control of T7-prmoter | |
| pMLBAD- | Trimethoprim resistant, expression of M. | This study |
| Mh.NGTmyc | haemolytica NGT with C-terminal c-myc- | |
| | tag under control of arabinose promoter | |
| pEC415-AP1697myc | Ampicillin resistant, expression of A. | This study |
| | pleuropneumonia NGT with C-terminal c- | |
| | myc-tag under control of tac promoter | |
| pMLBAD-AP1697myc | Trimethoprim resistant, expression of A. | This study |
| | pleuropneumonia NGT with C-terminal c- | |
| | myc-tag under control of arabinose | |
| | promoter | |
| pFLA91 | Ampicillin resistant, expression of A. | Schwarz et al., J Biol |
| | pleuropneumonia NGT with C-terminal | Chem. 2011 Oct. |
| | His₆ under control of arabinose promoter | 7; 286(40):35267-74 |
| pFLA100 | Ampicillin resistant, expression of A. | This study |
| | pleuropneumonia AtaC under control of | |
| | Pₐᵣₐ | |
| pAN15 | Trimethoprim resistant, expression of A. | This study |
| | pleuropneumonia AtaC with C-terminal | |
| | His₆ under control of Pₐᵣₐ | |

1-14. (canceled)

15. N-glycosylated protein for the manufacture of a medicament for treating and/or preventing bacterial Pasteurellaceae infection in a mammal or bird, wherein the protein is a Pasteurellaceae protein, a functional fragment or derivative thereof having at least one glycosylated N-X-S/T consensus sequence, wherein X is not Pro, and wherein the Pasteurellaceae protein, functional fragment or derivative thereof is
(1) autotransporter adhesin [ataC] A. pleuropneumoniae serotype 7 (strain AP76) (accession no. B3GX20_ACTP7; SEQ ID NO: 1), or
(2) AT family autotransporter/adhesin [COK_{—}1394] Mannheimia haemolytica serotype A2 str. BOVINE (accession number E2P8A5_PASHA; SEQ ID NO: 13), or
(3) a functional fragment or derivative having at least 40, preferably at least 50, more preferably at least 70, most preferably at least 80% amino acid sequence identity to (1) or (2).

16. N-glycosylated protein according to claim 15, wherein the N-linked glycan is selected from the group consisting of β-Glc/β-Gal, (β-Glc/β-Gal)-1,6-(α-Glc/α-Gal)ₙ, wherein n is at least 1, preferably 1 to 10, preferably 1 to 6, more preferably 2 to 5, more preferably β-Glc-α1,6-Glc-α1,6-Glc, β-Glc-α1,6-Glc-α1,6-Glc-α1,6-Glc, β-Glc-α1,6-Glc-α1,6-Glc-α1,6-Glc-α1,6-Glc, β-Glc-α1,6-Glc-α1,6-Glc-α1,6-Glc-α1,6-Glc-α1,6-Glc, β-Gal-α1,6-Glc-α1,6-Glc, β-Gal-α1,6-Glc-α1,6-Glc-α1,6-Glc, β-Gal-α1,6-Glc-α1,6-Glc-α1,6-Glc-α1,6-Glc and β-Gal-α1,6-Glc-α1,6-Glc-α1,6-Glc-α1,6-Glc-α1,6-Glc.

17. N-glycosylated protein according to claim 15, wherein the N-glycosylated protein is autotransporter adhesin [ataC] [A. pleuropneumoniae serotype 7 (strain AP76) (accession no. B3GX20_ACTP7; SEQ ID NO: 1), a functional fragment or derivative thereof having at least 40, preferably at least 50, more preferably at least 70, most preferably at least 80% amino acid sequence identity to ataC, wherein at least 10%, preferably at least 30%, more preferably at least 50%, most preferably at least 70% of all N-X-S/T consensus sequences are glycosylated.

18. N-glycosylated protein according to claim 17, wherein at least 10%, preferably at least 30%, more preferably at least 50%, most preferably at least 70% of all N-X-S/T consensus sequences are glucosylated, more preferably glucosylated by β-Glc-α1,6-Glc-α1,6-Glc.

19. N-glycosylated protein according to claim 15, wherein the N-glycosylated protein is
(i) a fragment of autotransporter adhesin [ataC] A. pleuropneumoniae serotype 7 (strain AP76) (accession no. B3GX20_ACTP7; SEQ ID NO: 1), preferably comprising amino acids 1866 to 2516 (SEQ ID NO: 16);
(ii) a fragment of autotransporter adhesin [ataC] A. pleuropneumoniae serotype 7 (strain AP76) (accession no. B3GX20_ACTP7; SEQ ID NO: 1), preferably comprising amino acids 61 to 984 (SEQ ID NO: 17);
(iii) a fragment of autotransporter adhesin [ataC] A. pleuropneumoniae serotype 7 (strain AP76) (accession no. B3GX20_ACTP7; SEQ ID NO: 1), preferably comprising amino acids 51 to 2428 (SEQ ID NO: 18);
(iv) a fragment of autotransporter adhesin [ataC] A. pleuropneumoniae serotype 7 (strain AP76) (accession no. B3GX20_ACTP7; SEQ ID NO: 1), preferably comprising amino acids 1866 to 2428 (SEQ ID NO: 19);
wherein (i), (ii), (iii) and (iv) are N-glycosylated, in
(a) 1 to 14, preferably at least 2 to 10, more preferably 2 to 8 consensus sequence(s) N-X-S/T for fragment (i),
(b) 1 to 9, preferably at least 2 to 8, more preferably 2 to 5 consensus sequence(s) N-X-S/T for fragment (ii),
(c) 1 to 73, preferably at least 2 to 50, more preferably 5 to 20 consensus sequence(s) N-X-S/T for fragment (iii) and
(d) 1 to 13, preferably at least 2 to 10, more preferably 2 to 8 consensus sequence(s) N-X-S/T for fragment (iv).

20. N-glycosylated protein according to claim 19, wherein the N-glycosylated protein is glucosylated, more preferably glucosylated by Glc-α1,6-Glc-α1,6-Glc.

21. Pharmaceutical composition comprising a pharmaceutically effective amount of at least one N-glycosylated protein according to claim 15 and optionally one or more pharmaceutically acceptable carriers and/or adjuvants.

22. Method for producing N-glycosylated proteins according to claim 15, comprising the following steps:
(i) providing a cell, preferably a prokaryotic cell, more preferably an E. coli cell expressing an N-glycosyltransferase (NGT) and a Pasteurellaceae protein, functional fragment or derivative thereof having at least one N-X-S/T consensus sequence(s), wherein X is not Pro;
(ii) culturing said cell under conditions that lead to the N-linked glycosylation, preferably glucosylation of said Pasteurellaceae protein,
(iii) optionally coexpressing an glycosyltransferase for glycosyl extension of N-linked glycosyl, preferably for extending glucosyl residues, and
(iv) optionally purifying the N-glycosylated proteins.

23. Method according to claim 22, wherein the glycosyltransferase in step (iii) is selected from a Pasteurellaceae protein, preferably proteins of Actinobacillus, Haemophilus, Histophilus and Mannheimia, more preferably proteins from Actinobacillus pleuropneumoniae, Haemophilus influenza, Haemophilus parasuis, Histophilus somni and Mannheimia haemolytica, most preferably an α6GlcT from A. pleuropneumoniae.

24. Method of diagnosing a bacterial Pasteurellaceae infection comprising the following steps:
(i) providing a sample, preferably selected from blood, saliva, lacrimal, urine and/or feces from a mammal or bird suspected of having a Pasteurellaceae infection,
(ii) contacting said sample, a functional component or derivative thereof with an N-glycosylated protein according to claim 15 under conditions that allow for antibody binding, and
(iii) determining binding of said sample, a functional component or derivative thereof to an N-glycosylated protein according to claim 15.

25. A method of treating and/or protecting mammals or birds having or being prone to develop a bacterial Pasteurellaceae infection comprising the administration of a therapeutically effective amount of at least one N-glycosylated protein according to claim 15 to a patient in need thereof.

26. The method of claim 24, wherein the N-glycosylated protein is a Pasteurellaceae protein, preferably a protein from the genera Actinobacillus, Aggregatibacter, Avibacterium, Basfia, Bibersteinia, Chelonobacter, Gallibacterium, Haemophilus, Histophilus, Lonepinella, Mannheimia, Nicoletella, Pasteurella, Phocoenobacter and Volucribater, more preferably a protein of Actinobacillus, Haemophilus, Histophilus and Mannheimia, most preferably a protein from Actinobacillus pleuropneumoniae, Haemophilus parasuis, Haemophilus influenza, Histophilus somni and Mannheimia haemolytica.

27. A method of treating and/or protecting mammals or birds having or being prone to develop a bacterial Pasteurellaceae infection comprising the administration of a therapeutically effective amount of at least one N-glycosylated protein according to a pharmaceutical composition of claim 21 to a patient in need thereof.


SEQUENCE LISTING (35 sequences)

SEQ ID NO: 1 - protein, 2516 aa, Actinobacillus pleuropneumoniae
MLHLDCKFSQLKEYVGNNSSYTWNISDNAEGTNSATVSNKDTVAVQGSVKKDESTTKSGIVTKLDGKNITVDLSEKAKQELENGQKHSSVNGDTNVLVEVNKAPNAEGGKQYDVKLADKIVIGQGDNSVTIDGTSGTVSGLNNLTWDPSATYEGGKAATQEQLKLVSDEVQKGWNVQTNNDTAQKVAPGETVKFVDGKNIKVTSNGKEITIATADNVVTTDTDKYVTGGKVEYDNQGNGTTTLTLRDGSTAQVTGAKNNFVTSAKTDPNGKKATLTRNDGGTVDIDLTNTVNQAVNEATEKGTHYEGDKPDTGATANNFKRKLGETTKVLGGAQGELSDNNIGVVSNGSDTLTVKLAKSLTDLTNATFGSNDDKTVINKDGVTITNGADANKTVSLTDGGLNNGGNKITNVAKGDADTDAVNVSQLNQAIKDSSYNWNISDGKTEQAVPDDGKVSVKGSANDDSKATSGIVTTLTGTDITVDLSDKSKQDIADGKKHSSVEGDANVVVTQTTTNKDGGKQYDVKLADKVSIGSDKATTVTIDGTKGTVSGLTNTTWDPNKEYTGGQAATQEQLKSVSDVANSGWNISANGQNESNVGPKGKVSFNNTDGNVLISKETTDNNVTFNLNNDLTVGGAGKDGKDGKDGTLSVKGADGKTGVTLNGKDGTIGLTGAPGKDGKDAQATIKVVDGTKGLDGNNGKDGESKTRIVYEKPNGGGTEEIATLNDGLNFVGDKGQVIQKKLNETLAIKGNLDAAAVVTDKNLRVDNDKDKNGELIIKMAKSLTDLTNATFSSDDSNTVIGGNGLTITPKAGDEVSLTDKGLNNGNNTIINVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVTLNGKDGSIGLTGPKGADGKDGANATISVVNGPVGVDGTDGKDGKDGMTRIVYTDPKGTTHNVSTLNDGLNFAGNQGDTIVKKLNETLTVKGALANTADASSENLRVDSQDGALVVKLAQNLANLTTATFGNTDTDKTVVSKDGVTISNGSDKDKTVSLTDKGLNNGNNQITNVTSGLTDSTGQKSDLANATTTNAVNVGDLKDTVNNLTNATTGGFGLKDDNNTEVKQDLGKTIQIKGKDGVTVTSNVADKSLEVALQGDVTVNGKDGKDGSIGVKGADGKDGTKITKDAVVFNGVDGKDGKDGQVSIKVEQGEKGIAGNDGANGTTKTRIVYEKPNGDKEQVATLNDGLNFVGDKGQVIQKKLNETLAIKGNLDANATVTDKNLRVDNDKDQNGELIIKMAKSLTDLTNATFSSGDINATIGGNGLTITPKGGDVVSLTDKGLNNGNNTITNVAPGVNGTDAVNKGQLDGVNATANAGWNLTTNGDNTNASNVAPNSTVDLANTDGNIVITKAGNNVTFDLNNNLTVGGPGKDGKDGVDGQLGVQGKDGKTGVALNGKDGAIGINGKDGSNGSITVKQGKPGVDGKDGETKTRIAYETKDETGKPTTEEVATLKDGLKFVGDTGEVIAKKLNETLAIKGNLTATAAVTDKNLRVDNENGQLIVKMAKSLTDLTNATFGSDNSNTTIGGNGVTITPKGGDASNTVSLTDKGLNNGNNQVTNVSTGLKDRDGNNVTLANASGDVLNNAVNVGDLKDSVNNLTNATTGGFGLTDEKGNDVKADLGKTVTVQGDGSVKTEVVEKDGKKALQIGLTNNVTVGNDKEPGTITVKGENGKDGVSISGKDGISIKGENGQDAVSINGKDGNGAIAVNGKDGKTGVGLDGANGTIGINGKDGSNGTITLAKGEPGVDGKDGKTRIVYEAKTPDGKTVTEEVATLKDGLKFVGNDGKVITKELNETLTIKGNLSTAADVTDKNLRVDNVDNALIIKMARTLTDLTSATFTNAGGDKSVVDGNGLTITPTKGGNTVSLTTSGLDNGGNKVINVAAGDVNANSTDAVNGSQLYAVSEVANKGWNIQTNGNDTTNVKPGDTVNFVNGDNIAITNDGTKVTVGLVKNVDLGEDGSIKAGDTFVNKDGVKVGDNVSLTKDGLTAGDVKISATTGINAGDKQITNVASGLGGKKLSEAEGDTLTNAANIGDLQTAVSSVTDASQGGGFGLADDKGANVTQNLGKTIAVKGDGKNISTVVKGGALTVNLNKDVDLGKDGSLTIGNTTINSDQVKVGDVTVSSNGKVSGVADGDISPNSTEAINGSQLYDANQNIANYLGGGSKLDDKGNVIAPTYTITKVDGNTTTANNVGDAITNLNNEVVKPLTFEGDTGVASKRKLGSTVTIKGGVQDSSKLSENNIGVVSDGKGTLAVKLAKDIKVDSVEAKTVNANTVNANTVKAGDTTINSDGVTIKDGPSVTKSGINAAGNRITNVKAGQADTDAVNVSQLKGAVGHVNQRINKVNKDLRAGIAGANAAAGLPQAYMPGKSMMAVAAGTYKNESALAVGYSRSSDNGKVILKLQGNANTRGDLGGSVGVGYQW

SEQ ID NO: 2 - protein, 3317 aa, Actinobacillus pleuropneumoniae
MQWTKTESTAGGAISVGYEAKSKALDAIAIGTTAKALTGTAIAIGLGATSSVDGGVALGANSISDRSMTVDATKKPFIPATATQEQEKAINDTVATTGAVSLGKDGVRRQITNLAAGAEDSDAVNVAQLKAAVTKAVASSTWNIQENTTQKEVVNSGDDVNFADGIGTTATVDVNKNASTSTSTSTVKFSVNQSDLNVANNGSVTAVTKGDYFATAEQVAKAINDSEKTTVINSSKTITAKPTVNGTVTTYDLDLSEGTKKDIEKGVDAHTTVTNKGITFTADSGTETLRKLGDTLAINGDKALIDTTVETGKVSVAATKKLQDAVASAESALQEIETTANGVKAQTIKKDSNKANFTNGKNIKLTPSTTGIEVATKDDVTFTTVNATTVNGDTIKSGNVVINKDGINAGNQKITNVAKGTEDSDAVNYSQLKEYVGNNSSYTWNISDNAEGTNSATVSNKDTVAVQGSVKKDESTTKSGIVTKLDGKNITVDLSEKAKQELENGQKHSSVKGDTNVVVTQTATNTEGGKQFDVKLADKVVIGQGDNSVTIDGTSGTVSGLNNLTWDPNATYEGGKAATQEQLKLVSDVAQKGWNVQTNNDAAQKVAPGETVKFKDGKNIKVTNIGKEITIATADNVVTTDTDKYVTGGSVTYNDKGDATATLTGNNGAGGAITGVKNNFVTKAETATDGKKATLTRNDDVTVDIDLTNTVNQAVNEATEKGTNYEGDKADTGVADNNFKRKLGETTKVLGGAQGELSDNNIGVVSNGKDTLTVKLAKTLSGLTSATFTSGDSNTTIDGNGLTITPNNGNEVSLTDKGLNNGDNRITNVAKGQDGTDAVNLSQLKEYVGDNSYNWNISDGEKTANVPDNNTVSVKGSVKTADSATENGIVTKLDGTDITVDLSDKAKQDIADGKKHSSVKGDANVLVEVNNTPNSDGGKQYDVKLADTVVVGKDKVDGKPVTIDGTTGEISGLTNTTWNPNETYTGGKSATQEQLKAVSDVAHLGWNVQTNDTLASKVAPGETVKFVDGKNIKVTSNGKDITIATADNVVTTETDKYVTGGKVEYDNQGNGTTTLTLRDGSTAQVTGAKNNFVTEAETAKDGKTATLTRNDGGKVNIDLTNTVNQSVTEATDKGTKYAGDKADTGAAANEFGRKLGETTNVKGGAQGELSDNNIGVVSNGVDTLTVKLAKALSGLTSATFGSDATDQTVINKDGITINADKPENTVTLTDNGLNNGGNKITNVAKGDADTDAVNVSQLNQAIKDSAYNWNISDGKTEQAVPDNGKVSVKGSANDDSKATSGIVTILTGTDITVDLSDKSKQDIADGKKHSSVEGDANVVVTQTTTNKDGGKQYDVKLADKISIGSDKATTVTIDGTKGTVSGLTNTTWDPNKEYTGGQAATQEQLKSVSDVANSGWNISANGQNESNVGPKGKVSFNNTDGNVLISKETTDNNVTFNLNNDLTVGGPAGKDGKDGKDGSIGVKGADGKTGITLNGKDGSIGLTGAPGKDGQNAQATIKVVDGTKGLDGNNGKDGESKTRIVYEKPNGGGKEEIATLNDGLNFVGDKGQVIRKKLNDTLAIKGNLDAAAVVTDKNLRVDNDKDQNGELIIKMAKSLTDLTNATFSSDDSNTVIGGNGLTITPKTGDAVSLTDKGLNNGNNTITNVKAGENDTDAVNVSQLKEVKNTANAGWNISANGQNETNVAPKGSVSFNNTDGNVVITKAADNNNVTFNLNNDLTVGGPAGKDGKDGKDGTIGVKGKDGTTGVTLNGKDGSIGLTGAKGADGKDGANATITVANGPVGVDGTDGKDGKDGMTRIVYTDPKGTTHNVSTLNDGLNFAGNQGDTIAKKLNETLTVKGDLANDAAASSENLRVDSQDGALVVKLTQNLANLTTATFGNTDTDKTVVSKDGVTISSDKPEKEVSLTDKGLNNGNNQITNVTSGLTDSTGQKSDLANATTTNAVNVGDLKDTVNNLTNATTGGFGLKDDNNTEVKQDLGKIIQIKGKDGVTVTSDVANKSLEVALQGDVTVNGKDGKDGSIGVKGADGKDGTKITKDAVVFNGVDGKDGKDGQVSIKVEQGPKGIAGNDGKDGESKTRIVYEKPNGDKEQVATLNDGLNFVGDKGQVIQKKLNETLAIKGNLDANATVTDKNLRVDNDKDQNGELIIKMAKSLTDLTNATFSSGDINATIGGNGLTITPKGGDVVSLTDKGLNNGNNTITNVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVALNGKDGTIGINGKDGSNGSITVKQGKPGVDGKDGETKTRIVYETKDETGKPTTEEVATLKDGLKFVGDTGEVIAKKLNETLAIKGNLTATAAVTDKNLRVDNENGQLIVKMAKSLTDLTNATFGSDNSNTTIGGNGVTITPKGGDASNTVSLTDKGLNNGNNQVTNVSTGLKDRDGNNVTLANASGDVLNNAVNVGDLKDSVNNLTNATTGGFGLTDEKGNDVKADLGKTVTVQGDGSVRTTVVEVEKDGKKEKVLEVGLTNNVTVGNDKEPGTITVKGENGKDGVSISGKDGIGIKGENGQDAVSINGKDGDGTVAVKGKDGKTGVALNGKDGTIGINGKDGSNGSITVAQGEKGLDGNDGANGKTKTRIVYEKPNGDKEEVATLNDGLNFVGNDGKVIAKKLNKTLAIKGNLSTAVKDVTANNLRVDNVGDELIINMAKALTDLTSATFTNKDGNKSVVDGNGLTITPTKGGNIVSLTTSGLDNGGNKVINVAAGDVNANSTDAVNGSQLYAVSEVANKGWNIQTNGNDTTNVKPGDTVNFVNGKNIEINNDGTNVTVGLAKDVDLGKDGSIKAGDTIMNNEGVKVGDNVSLTKDGLKAGNVTISATTGINAGDKQITNVASGLGGKKLSEAKGDTLTNAANIGDLQTAVSSVTDASQGGGFGLADDKGQEVKQNLGKTIPVKGDGKNIITVVKGGALTVNLNKDVDLGKDGSVTTGNTKVSDKGVSFADSLVNLTSNGLDNGGNKVTNVKAGDVNANSTDAVNGSQLYATNQNVTNVQNEVAKGWNIEAGTVDGGKVFNASKTKVAMGDTVGVKAGKNIEITQDGSNIAIATSANPTFETVTTESVKVGKGDNTVAIETVTDKHGSALKVSGADGKSETRINNVADGKADNDAVNVRQLRGVAQNVANIDNRVSKLDKRVRGIGANAAAASSLPQVYIPGKSMVALAGGAYSGASAVAVGYSRASDNGKVILKVNGTANSAGHYSGGVGVGYQW

SEQ ID NO: 3 - protein, 2974 aa, Actinobacillus pleuropneumoniae
MAIGTHAVVTKADAVAIGHNSGAVNVNTTAIGSGANASGERSTALGNKASSSGNNATALGVEANASGQESNVLGYKANSNGYRAMALGTLSNAAGTMAIALGSQSNASGIKAQAFGSNAIASAEDAIAMGSNANANAVSGVALGNRSMASGVSGVALGDGAASNAQQSISIGKSARTTATNSIALGNASLATADSAIAMGNSSSAVGSNGIAVGAKSKALKDNAIALGNNATVTANNALALGLNSVAYSDNAIVLGTTSKATGSHSVAIGTGASAYRQNSIAMGKDSSTGGDFAVALGDSANAAAENTLALGKNAIADKKDSVALGNNAYTGDVIATESATLAGKEYKFAGTAPVGTVSIGSEGKERTITNVAAGRISSTSTDAINGSQLYAVSEVASRGWNIQANGDTASQVAPGATVQFIDGTNIDITRDGNNITIATATNVVTTDTDKYVTGGTVVYNDKGEATATLTGNNGAGGAITGVKNNFVTKSETATDGKKATLTRNDGGTVDIDLTNTVNQAVTEATDKGTKYAGDKADTGAAANEFGRKLGETTNVKGGAQGELSDNNIGVVSNGVDTLTVKLAKALSGLTSATFGSDATDQTVINKDGVTINSTTPDKTVSLTETGLNNGGNKITNVAEGKAGTDAVNVNQLNQAITNNAYNWNISDGTNNTAVPDSGTVAVKGSANADSANTVGVVTELEGTDVKVDLSQKAKDDIANGQKHSSVAGDTNFVVTQTTTNAEGGKQYDVKLADTVVIGKDKTNGGNPVTINGTTGEISGLTNTTWDPDTKYEGGKAATQEQLKSVSDVAQKGWNIQANKDTATKVAPGDTVKFIDGENIKITRTGNDITVATAKDVKFDSVKVGDKVSINNDGINAGDTKVTNVTNGTLAADSKDAVNGSQLYATNQNVTNNAANITKNADNIAKGTVYAGDRLDATVTGKTNNFTRALGEQTNVVGGATGELSDNNLAVISNGTDTLTVKLAKSLTDLTNATFGSTDTDKTVINKDGVTVSSDKPEKEVSLTDKGLNNGNNQITNVTSGLVKRDGNPVELSKAEGDVLTNAVNVGDLKTTVTNLTEEGKGGGFGLTAEDNKDVKADLGKTVKVQGDGSVKTTVVEKDGQSALQVGLTENVTVGGQDKPGTITVKGENGKDGVSINGKDGSIGLTGAPGKDGKDAQATIKVVDGTKGLDGNNGKDGESKTRIVYEKPNGGGTEEIATLNDGLNFVGDKGQVIQKKLNETLAIKGNLDAAAVVTDKNLRVDNDKDKNGELIIKMAKSLTDLTNATFSSDDSNTVIGGNGLTITPKAGDEVSLTDKGLNNGNNTIINVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVTLNGKDGSIGLTGPKGADGKDGANATISVVNGPVGVDGTDGKDGKDGMTRIVYTDPKGTTHNVSTLNDGLNFAGNQGDTIVKKLNETLTVKGALANTADASSENLRVDSQDGALVVKLAQNLANLTTATFGNTDTDKTVVSKDGVTISNGSDKDKTVSLTDKGLNNGNNQITNVTSGLTDSTGQKSDLANATTTNAVNVGDLKDTVNNLTNATTGGFGLKDDNNTEVKQDLGKTIQIKGKDGVTVTSNVADKSLEVALQGDVTVNGKDGKDGSIGVKGADGKDGTKITKDAVVFNGVDGKDGKDGQVSIKVEQGEKGIAGNDGANGTTKTRIVYEKPNGDKEQVATLNDGLNFVGDKGQVIQKKLNETLAIKGNLDANATVTDKNLRVDNDKDQNGELIIKMAKSLTDLTNATFSSGDINATIGGNGLTITPKGGDVVSLTDKGLNNGNNTITNVAPGVNGTDAVNKGQLDGVNATANAGWNLTTNGDNTNASNVAPNSTVDLANTDGNIVITKAGNNVTFDLNNNLTVGGPGKDGKDGVDGQLGVQGKDGKTGVALNGKDGAIGINGKDGSNGSITVKQGKPGVDGKDGETKTRIAYETKDETGKPTTEEVATLKDGLKFVGDTGEVIAKKLNETLAIKGNLTATAAVTDKNLRVDNENGQLIVKMAKSLTDLTNATFGSDNSNTTIGGNGVTITPKGGDASNTVSLTDKGLNNGNNQVTNVSTGLKDRDGNNVTLANASGDVLNNAVNVGDLKDSVNNLTNATTGGFGLTDEKGNDVKADLGKTVTVQGDGSVKTEVVEKDGKKALQIGLTNNVTVGNDKEPGTITVKGENGKDGVSISGKDGISIKGENGQDAVSINGKDGNGAIAVNGKDGKTGVGLDGANGTIGINGKDGSNGTITLAKGEPGVDGKDGKTRIVYEAKTPDGKTVTEEVATLKDGLKFVGNDGKVITKELNETLTIKGNLSTAADVTDKNLRVDNVDNALIIKMARTLTDLTSATFTNAGGDKSVVDGNGLTITPTKGGNTVSLTTSGLDNGGNKVINVAAGDVNANSTDAVNGSQLYAVSEVANKGWNIQTNGNDTTNVKPGDTVNFVNGDNIAITNDGTKVTVGLVKNVDLGEDGSIKAGDTFVNKDGVKVGDNVSLTKDGLTAGDVKISATTGINAGDKQITNVASGLGGKKLSEAEGDTLTNAANIGDLQTAVSSVTDASQGGGFGLADDKGANVTQNLGKTIAVKGDGKNISTVVKGGALTVNLNKDVDLGKDGSVTTGNTKVSDKGVSFADSLVNLTSNGLDNGGNKVTNVKAGDVNANSTDAVNGSQLYATNQNVTNVQNEVAKGWNIEAGTVDGGKVFNASKTKVAMGDTVGVKAGKNIEITQDGSNIAIATSANPTFETVTTESVKVGKGDNTVAIETVTDKHGSALKVSGADGKSETRINNVADGKADNDAVNVRQLRGVAQNVANIDNRVSKLDKRVRGIGANAAAASSLPQVYIPGKSMVALAGGAYSGASAVAVGYSRASDNGKVILKVNGTANSAGHYSGGVGVGYQW

SEQ ID NO: 4 - protein, 2323 aa, Actinobacillus pleuropneumoniae
MNQAITNNAYNWNISDGTNNTAVPDSGTVAVKGSANADSANTVGVVTELEGTNVKVDLSQKAKDDIANGQKHSSVAGDTNFVVTQTTTNAEGGKQYDVKLADTVVIGKDKTNGGNPVTINGTTGEISGLTNTTWDPDTKYEGGKAATQEQLKSVSDVAQKGWNIQANKDTATKVAPGDTVKFIDGENIKITRTGNDITVATAKDVKFDSVKVGDKVSINNDGINAGDTKVTNVTNGTLAADSKDAVNGSQLYATNQNVTNNAANITKNADNIAKGTVYAGDRLDATVTGKTNNFTRALGEQTNVVGGATGELSDNNLAVISNGTDTLTVKLAKSLTDLTNATFGSTDTDKTVINKDGVTVSSDKPEKEVSLTDKGLNNGNNQITNVTSGLVKRDGSSVELSKAEGDVLTNAVNVGDLKTTVTNLTEEGKGGGFGLTAEDNKDVKADLGKTVKVQGDGSVKTTIVEKDGQSALQVGLTENVTVGGQDKPGTITVKGENGKDGVAINGKDGSIGLTGAPGKDGQDAQATIKVVDGTKGLDGNNGKDGESKTRIVYEKPNGGGTEEIATLNDGLNFVGDKGQVIRKKLNETLAIKGNLDANATVTDKNLRVDNDKDQNGELIIKMAKSLTDLTNATFSSDDSDTVIGGNGLTITPKAGNAVSLTDKGLNNGNNTITNVKAGENDTDAVNVSQLKEVKNTANAGWNISANGQNETNVAPKGSVSFNNTDGNVVITKAADNNNVTFNLNNDLTVGGPAGKDGKDGKDGTIGVKGKDGTTGVTLNGKDGSIGLTGAKGADGKDGANATITVANGPVGVDGTDGKDGKDGMTRIVYTDPKGTTHNVSTLNDGLNFAGNQGDTIAKKLNETLTVKGDLANDAAASSENLRVDSQDGALVVKLAQNLANLTTATFGNTDTDKTVVSKDGVTISSDKPEKEVSLTDKGLNNGNNQITNVTSGLTDSTGQKSDLANATTTNAVNVGDLKDTVNNLTNATTGGFGLKDDNNTEVKQDLGKTIQIKGKDGVTVTSDVANKSLEVALQGDVTVNGKDGKDGTIGVKGADGKDGTKITKDAVVFNGVDGKDGKDGQVSIKVEQGEKGIAGNDGANGTTKTRIVYEKPNGDKEQVATLNDGLNFVGDKGQVIRKKLNETLAIKGNLDANATVTDKNLRVDNDKDQNGELIIKMAKSLTDLTNATFSTGDINATIGGNGLTITPKDGDVVSLTDKGLNNGNNTITNVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVALNGKDGTIGINGKDGSNGSITVKQGKPGVDGKDGETKTRIVYETKDETGKPTTEEVATLNDGLKFVGDTGEVIAKKLNETLAIKGNLTATTAVTDKNLRVDNENGQLIVKMAKSLTDLTNATFGSDNSNTVIGGNGVTITPNGGDAGNTVSLTDKGLNNGNNQVTNVSTGLKDRDGNNVTLANASGDVLNNAVNVGDLKDSVNNLTNATTGGFGLTDEKGNDVKADLGKTVTVRGDGSVKTEVVEKGGKKALQIGLTNNVTVGNDKEPGTITVKGENGKDGVSISGKDGIGIKGENGQDAVSINGKDGDGTVAVKGKDGKTGVALNGKDGTIGINGKDGSNGTITLAKGEPGVDGKDGKTRIVYETKTPDGKTVTEEVATLKDGLKFVGNDGKVITKELNETLTIKGNLSTAADVTDKNLRVDNVDNALIIKMARTLTDLTNATFTNAGGDKSVVDGNGLTITPSGNASNTVSLTTTGLNNGDNKVINVAAGDVNATSTDAVNGSQLYAVSEVANKGWNIQTNGNVSTNVKPGDTVNFANGKNIEINNDGTNVTVGLAKDVDLGKDGSIKVSDTIMNNEGVKVGDNVSLTKDGLKAGNVTISATTGINAGDKQITNVASGLGDKKLSEAKGDTLTNAANIGDLQTAVSSVTDASQGGGFGLADDKGANVTQNLGKTIAVKGDGKNISTVVKGGALTVNLNKDVDLGKDGSVTTGNTKVSDKGVSFADSLVNLTSNGLDNGGNKVTNVKAGDVNANSTDAVNGSQLYATNQNVTNVQNEVAKGWNIEAGTVDGGKVFNASKTKVAMGDTVGVKAGKNIEITQDGSNIAIATSANPTFETVTTESVKVGKGDNTVAIETVTDKHGSALKVSGADGKSETRINNVADGKADNDAVNVRQLRGVAQNVANIDNRVSKLDKRVRGIGANAAAASSLPQVYIPGKSMVALAGGAYSGASAVAVGYSRASDNGKVILKVNGTANSAGHYSGGVGVGYQW

SEQ ID NO: 5 - protein, 3347 aa, Actinobacillus pleuropneumoniae
MNKIFKVIWSHATQTFVVVSELTRSKSKAKASVSDVKKNEVSSLITSGFKLSAISSLLISTIPAAYAAVAINNTQGSDINTNGVQVTGSGIAIGSSAGYQQTDGYTSNNSAEAKRNVYKLQDIGGTASNNTVSLGTNAYAGGEDSVAIGTRAQAVHNVQTTDMAYNSRGSYNAGAVAIGFQSIARGDQTVALGSRAAAYNRQATAIGNDSYAMGVGSVVIGGDDSGYAYESINGSHNSPKRPWANNEGNLGYTNLATGFNPSNELNKADYRPSAASGNGATVVGVHSQALSKGSTALGVAATAGDGGKTSSTIKDGQIDGIDRTQNIEATAIGALAHARDRNTTAVGHAAKAYSEYSTSIGDDAVAKGKNSSAVGKSSYAQGNYSVAIGNAEDTTNADRVNATKVIANGTRAIAIGTTARTNADDTIAFGTRTESTAGGAISIGYEAKSKALDAIAIGKGAKALNTNTISIGTGNTVNGANSGAIGDPTVVNGTNSYSLGNNNTIDSNNTFVLGNSVKLNTNAKNSTVIGNQNTINLTDVATNNVNTGYVNIQGDHNTINAARGIGILGNENTVDGVTHDVFILGNNVKKTANDSVFLGSGSGYVAEDTTTKGLSKIYTSDTVNGKPVNFAGGNVTAGVVSVGGVNEKDEKSTRRIQNVAPGLISAQSTDAINGSQLYAVADALKTNIPESKWNIKENSTQKDVVNSGDNVSFANGNGTTANVSVSQDETTSTVKYSVNQSNLNVAQDGEVTAANKGDNFATADQVAQAINNSEKTTVINNGTTITATPTTTGTVTTYDLDLSKTTKDDIKQGVDANTTVNTKGITFTADNAGAKTERKLGETLAINGDKALINTTVEAGKVSVAATKKLQDAVVSAESALQEIVTTVNGTTAQTVNKNSNKANFINGKNIELTPSTTGITVATKENVDFTTVNATTVNATTVNGDTIKSGNVVINQDGINAGDKKITHVADGNVTNDSKEAVNGSQLYATNQNVTNNTNNIAKNTADIAKGTVYAGDYAKDDSVKNEFTQALGNQTNVVGGASKDDLSDNNIGVVSDGVTTLTVKLAKTLEGLTSATFGNDANDHTVINKDGVTITNSTDPNKTVSLTDGGLNNGGNKITNVAEGKAGTDAVNVDQLTKAITDSAYNWNISDGTNNTAVPDSGTVAVKGSANADSATKSGIVTNLTGTDITVDLSEKAKQELENGQKHSSVEGDSNVLVNVNATLNAAGGTQYDVKLADTVVIGQGDNSVTIDGTSGTVSGLNNLTWDPTENYTGGKAATQEQLKLVSDEVQKGWNVQTNNDPAQKVAPGETVKFKDGKNIKVTNTGKEITIATADNVVTTDTDKYVTGGTVVYNDKGEATATLTGNNGAGGAITGVKNNFVTSATTDANGKKATLTRNDGGTVDIDLTNTVNQAVTEATDKGTKYAGDKADTGAAANEFGRKLGETTNVKGGAQGELSDNNIGVVSNGVDTLTVKLAKALSGLTSATFGSDATDQTVINKDGVTINSTTPDKTVSLTETGLNNGGNKITNVAEGKAGTDAVNVDQLTKAITDSAYNWNISDGTNNTAVPDSGTVAVKGSANADSVNTVGVVTELEGTNVKVDLSQKAKDDIANGQKHSSVAGDTNFVVTQTTTNPEGGKQYDIKLADKVVIGKDKPVTIDGTNGTVSGLTNTTWDPNTTYTGGQAATQEQLKSVSDVVQNGWNIQANTDTATKVAPGDTVKFIDGENIKITRAGNDITVATAKDVKFDSVKVGDKVSINNDGINAGDTKVTNVTNGTLAADSKDAVNGSQLYATNQNVTNNAANITKNADNIAKGTVYAGDRLDAAVTGKTNNFTRALGEQTNVVGGATGELSDNNLAVISNGTDTLTVKLAKSLTDLTNATFGSTDTDKTVINKDGVTISSGKPEKEVSLTDKGLNNGNNQITNVTSGLVKRDGSSVELSKAEGDVLTNAVNVGDLKTTVTNLTEEGKGGGFGLTAEDNKDVKADLGKTVKVQGDGSVKTTIVEKDGQSALQVGLTENVTVGGQDKPGTITVKGENSKDGVAINSKDGSIGLTGAPGKDGQDAQATIKVVDGTKGLDGNNGKDGESKTRIVYEKPNGGGTEEIATLNDGLNFVGDKGQVIQKKLNETLAIKGNLDVNATVTDKNLRVDNDKDQNGELIIKMAKSLTDLTNATFSSDDSNTVIGGNGLTITPKAGDEVSLTDKGLNNGNNTIINVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVALNGKDGTIGINGKDGSNGSITVKQGKPGVDGKDGETKTRIVYETKDETGKPTTEEVATLNDGLKFVGDTGEVIAKKLNETLAIKGNLTATAAVTDKNLRVDNENGRLIVKMAKSLTDLTNATFGSDNSNTTIGGNGVTITPKGGDASNTVSLTDKGLNNGNNQVTNVSTGLKDRDGNNVTLANASGDVLNNAVNVGDLKDSVNNLTNATTGGFGLTDEKGNDVKADLGKTVTVQGDGSVKTEVVEKDGKKALQIGLTNNVTVGNDKEPGTITVKGENGKDGVSISGKDGISIKGENGQDAVSINGKDGNGAIAVNGKDGKTGVGLDGANGTIGINGKDGSNGTITLAKGEPGVDGKDGKTRIVYETKTPDGKTVTEEVATLKDGLKFVGDDGKIITKELNETLTIKGNLSTTAAVTDKNLRVDNVDGALIIKMARTLTDLTNATFTNAGGDKSVVDGNGLTITPINGGKTVSLTTKGLDNGGNKVINVAAGDVNATSTDAVNGSQLYAVSEVANKGWNIQTNGSNTTNVKPGDTVNFANGDNIAITNDGTKVTVGLAKDVDLGEDGSIKAGDTLVNNEGVKVGDNVSLTKDGLKAGNVTISATTGINAGDKQITNVASGLGGKKLSEAEGDTLTNAANIGDLQTAVSSVTDASKGGGFGLADDKGANVTQNLGKTIAVKGDGKNISTVVKGGALTVNLNKDVDLGKDGSLTIGNTTINSDQVKVGDVTVSSNGKVSGVADGDISPNSTQAINGSQLYDANQNIANYLGGGSKLDDKGNVIAPTYTITKVDGNTTTANNVGDAITNLNNEVVKPLTFEGDTGVASKRKLGSTVTIKGGVQDSSKLSENNIGVVSDGKGTLAVKLAKDIKVDSVEAKTVNANTVNANTVKAGDTTINSDGVTIKDGPSVTKSGINAAGNRITNVKAGQADTDAVNVSQLKGAVGHVNQRINKVNKELRAGIAGANAAAGLPQAYIPGKSMMAVAAGTYKNESALAVGYSRSSDNGKVILKLQGNANTRGDLGGSVGVGYQW

SEQ ID NO: 6 - protein, 1404 aa, Actinobacillus pleuropneumoniae
MTSGLTDSTGQKSDLANATTTNAVNVGDLKDTVNNLTNATTGGFGLKDDNNTEVKQDLGKIIQIKGKDGVTVTSDVANKSLEVALQGDVTVNGKDGKDGSIGVKGADGKDGTKITKDAVVFNGVDGKDGKDGQVSIKVEQGPKGIAGNDGKDGESKTRIVYEKPNGDKEQVATLNDGLNFVGDKGQVIQKKLNETLAIKGNLDANATVTDKNLRVDNDKDQNGELIIKMAKSLTDLTNATFSSGDINATIGGNGLTITPKGGDVVSLTDKGLNNGNNTITNVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVALNGKDGTIGINGKDGSNGSITVKQGKPGVDGKDGETKTRIVYETKDETGKPTTEEVATLKDGLKFVGNDGKVVTKELNETLAIKGGINTEAGLTAASDRNVGVRENEKGLNIVIAERPTFSGITVDGKDGKDAEVKFAKDGKDGMSIVGTRGADGQNGLTLKGANGKDGVSFKEDGRITNVADGKDGKDAVNKDQLERVNATANAGWKLTINNGNNQTTVTPNATVDLANTDGNIVITKVGNNVNFGLNNTLTVGNDNKPGTMTVKGENGKDGVSISGKDGISIKGENGQDAVSINGKDGNGAIAVNGKDGKTGVGLDGANGTIGINGKDGSNGTITLAKGEPGVDGKDGKTRIVYETKTPDGKTVTEEVATLKDGLKFVGDDGKIITKELNETLTIKGNLSTTAAVTDKNLRVDNVDGALIIKMARTLTDLTNATFTNAGGDKSVVDGNGLTITPINGGKTVSLTTKGLDNGGNKVINVANGTISATSTDAVNGSQLYAVSEVANKGWNIQTNGNDTTNVKPGDTVNFVNGDNIAITNDGTKVTVGLVKNVDLGKDGSIKAGDTFVNKDGVKVGDNVSLTKDGLKAGNVTISATTGINAGDKQITNVASGLGGKKLSEAKGDTLTNAANIGDLQTAVSSVTDASQGGGFGLADDKGQEVKQNLGKTIPVKGDGKNISTVVKDGALTVNLNKDVDLGNDGSLTIGNTTINSDQVKVGDVTVSSNGKVSGVADGDISPNSTQAINGSQLYDANQNIANYLGGGSKLDDKGNVIAPTYTITKVDGNTTTANNVGDAITNLNNEVVKPLTFEGDTGVASKRKLGSTVTIKGGVQDSSKLSENNIGVVSDGKGTLAVKLAKDIKVDSVEAKTVNANTVNANTVKAGDTTINSDGVTIKDGPSVTKSGINAAGNRITNVKAGQADTDAVNVSQLKGAVGHVNQRINKVNKDLRAGIAGANAAAGLPQAYMPGKSMMAVAAGTYKNESALAVGYSRSSDNGKVILKLQGNANTRGDLGGSVGVGYQW

SEQ ID NO: 7 - protein, 3154 aa, Actinobacillus pleuropneumoniae
MNKIFKVIWSHVTQTFVVVSELTRSKSKAKASVSDVKKNGVSSLITLGLKLSLMSSLLSPSISTVYAAITIDKMDSYRFRDGTTINYATNSGTAKAGTGGSFSAYNYENPGNMNYEKQNNATVYSGTIKASSGIAIGENATTETTIGGKVAYSSGVAIGDFSKATGGLSFALGAYSQATDIGATVIGTAGLASGFNSLAMMRQSAAKANYAMAIGTASWADATASLAMGASATAKGVQSIAIGSSDMLAQDGGNGRALTKYDGTNNTEASGARSVAIGTTARTSADDAIAIGTGAKALNTNTISIGTGNTVTGANSTAIGGPTTISGTGSHSLGNDNTIPDDNSTVIGNNNKLSEGDSGQSTSDGSHVVGNNVQIRGENSLAFGNNAMVGDANNEGVSGTVNATAIGTSATAKGENALAVGTGAKASKTNNTAIGTSATITKDAGEDNIAFGSGATVTRGKNSIVMGTNSKSEATNGSSIAIGDGATIGNGVTSESVIPETQTSSTETADDGTTTQTDTATDGLDSNEVSSQGMTVSTSTTEVKTTTVTPKDGTTGPKITTVETDTVKTTGVDNDGIRINTTKIVTTTTPDGPTTVVENGEVSLTSGGLNNGGNRAINIAAGVNATDAVNVSQLEAAKTHYYSVKTTTEQANFNNDTATGENSIAAGPNVWAQGVQSAVFGSPAGASSDKATAVGNDAWAPGVQSTAIGSGVVAGGKGSVVIGSAENTEKTVPTAEAENRVAIGTHAVVTKADAVAIGHNSGAVNVNTTAIGSGANASGEHSTALGNKASSSGNNATALGVEANASGQESNVLGYKANSNGYRAMALGTLSNAAGTMAIALGSQSNASGIKAQAFGSNAIASAEDAIAMGSNANANAVSGVALGNRSMASGVSGVALGDGAASNAQQSISIGKSARTTATNSIALGNASLATADSAIAMGNSSSAVGSNGIAVGAKSKALKDNAIALGNNATVTANNALALGLNSVAYSDNAIVLGTTSKATGSHAVAIGTGASAYRQNSIAMGKDSSTGGDFAVALGDSANAAAENTLALGKNAVADKKDSVALGNNAYTGDVIATESATLAGQVYEFAGKAPIGTVSVGNQGNERTITNVAAGRISSTSTDAINGSQLYAVSEVASRGWNIQANGDKASQVAPGASVQFIDGTNIDITRDGNNITIATAANVVTTDTDKYVTGGKVAYDNQGNGTTTLTLKDGTEAQVTGAKNNFVTKSETATDGKKATLTRNDGGTVDIDLTNTVNQAVTEATDKGTKYAGDKADTGAAANEFGRKLGETTNVKGGAQGELSDNNIGVVSNGVDTLTVKLAKALSGLTSATFGSDATDQTVINKDGVTINSTTPDKTVSLTETGLNNGGNKITNVAEGKAGTDAVNVDQLTKAITDSAYNWNISDGTNNTAVPDSGTVAVKGSANADSVNTVGVVTELEGTNVKVDLSQKAKDDIANGQKHSSVAGDTNFVVTQTTTNPEGGKQYDIKLADKVVIGKDKPVTIDGTNGTVSGLTNTTWDPNTTYTGGQAATQEQLKSVSDVVQNGWNIQANTDTATKVAPGDTVKFIDGENIKITRAGNDITVATAKDVKFDSVKVGDKVSINNDGINAGDTKVTNVTNGTLAADSKDAVNGSQLYATNQNVTNNAANITKNADNIAKGTVYAGDRLDAAVTGKTNNFTRALGEQTNVVGGATGELSDNNLAVISNGTDTLTVKLAKSLTDLTNATFGSTDTDKTVINKDGVTISSGKPEKEVSLTDKGLNNGNNQITNVTSGLVKRDGSSVELSKAEGDVLTNAVNVGDLKTTVTNLTEEGKGGGFGLTAEDNKDVKADLGKTVKVQGDGSVKTTIVEKDGQSALQVGLTENVTVGGQDKPGTITVKGENSKDGVAINSKDGSIGLTGAPGKDGQDAQATIKVVDGTKGLDGNNGKDGESKTRIVYEKPNGGGTEEIATLNDGLNFVGDKGQVIQKKLNETLAIKGNLDVNATVTDKNLRVDNDKDQNGELIIKMAKSLTDLTNATFSSDDSNTVIGGNGLTITPKAGDEVSLTDKGLNNGNNTIINVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVALNGKDGTIGINGKDGSNGSITVKQGKPGVDGKDGETKTRIVYETKDETGKPTTEEVATLNDGLKFVGDTGEVIAKKLNETLAIKGNLTATAAVTDKNLRVDNENGRLIVKMAKSLTDLTNATFGSDNSNTTIGGNGVTITPKGGDASNTVSLTDKGLNNGNNQVTNVSTGLKDRDGNNVTLANASGDVLNNAVNVGDLKDSVNNLTNATTGGFGLTDEKGNDVKADLGKTVTVQGDGSVKTEVVEKDGKKALQIGLTNNVTVGNDKEPGTITVKGENGKDGVSISGKDGISIKGENGQDAVSINGKDGNGAIAVNGKDGKTGVGLDGANGTIGINGKDGSNGTITLAKGEPGVDGKDGKTRIVYETKTPDGKTVTEEVATLKDGLKFVGDDGKIITKELNETLTIKGNLSTTAAVTDKNLRVDNVDGALIIKMARTLTDLTNATFTNAGGDKSVVDGNGLTITPINGGKTVSLTTKGLDNGGNKVINVAAGDVNATSTDAVNGSQLYAVSEVANKGWNIQTNGSNTTNVKPGDTVNFANGDNIAITNDGTKVTVGLAKDVDLGEDGSIKAGDTLVNNEGVKVGDNVSLTKDGLTAGDVKISATTGINAGNKQITNVASGLGGKKLSEAEGDTLTNAANIGDLQTAVSSVTDASQGGGFGLADDKGQEVKQNLGKTIPVKGDGKNIITVVKDGALTVNLNKDVDLGKDGSVTTGNTKVSDKGVSFADSLVNLTSNGLDNGGNKVTNVKAGDVNANSTDAVNGSQLYATNQNVTNVQNEVAKGWNIEAGTVDGGKVFNASKTKVAMGDTVGVKAGKNIEITQDGSNIAIATSANPTFETVTTESVKVGKGDNTVAIETVTDKHGSALKVSGADGKSETRINNVADGKADNDAVNVRQLRGVAQNVANIDNRVSKLDKRVRGIGANAAAASSLPQVYIPGKSMVALAGGAYSGASAVAVGYSRASDNGKVILKVNGTANSAGHYSGGVGVGYQW

SEQ ID NO: 8 - protein, 3992 aa, Actinobacillus pleuropneumoniae
MMRQSAAKANYAMAIGTASWADATASLAMGASATAKGVQSIAIGSSDMLAQDGGNGRALTKYDGTNNTEASGARSVAIGTTARTSADDAIAIGTGAKALNTNTISIGTGNTVTGANSTAIGDPTNISGSDSHSLGNNNIIPDDNVTVIGNNNKLSEGDSGQSTSDGSHVVGNNVQIRGENSLAFGNNAMVGDANNEGVPGTVNATAIGTSATAKGKNALAVGTGAKASKTNNTAIGTSATITKDAGEDDIAFGSGATVTRGKNSIVMGKNAKSESTGNSSIVIGDGSSVIHGNNNIVMGTNSKSEATNGSSIAIGENATISSAVTGNAYVIGTNASIASTNGGIAFGNGAKITNSNGGNGTSVAIGEGAVATGTRAISLGYKAGNGGSQVDSIAIGSSATTSGINNIAIGKEAKASGSQGNQTVVGTGANADGDKSIVLGTNATAQGNQAEQIVIGTGANASGTSQYSIVMGSGAGATRENSTVLGSKANSIVDGGVALGANSVSDRQADGSATGHRSTYEPYIPVTAGQAQKDAINATKGTTGAVSVGSSTVKRQITNLAAGAEDTDAVNVAQLKAAVTTAVASSTWNIKENTTQKDVVNSGDNVSFDNGTGTTANVSVSQDGKTSTVKYSVNKSNLNVASNGTVTATNTGDNFATADQVAKAINDSEKTSSVVSTTQAIKVTSKVDGKNTEYDLDLSDATKTSLQNANSALQSWTAQANGNTVKTVSEDNATLNFVNGTNIEITNDSGQIKVATVASPTFTTVTADTVNAENFTTGTVSITDNGINAGNQKITNLANGTANSDAVTLAQLNASKSVVEAGNNTNVRSETATDGSKIYTVDANATTVSGSDALNITKSDAGNNVTNYALDLSDKTKGEIKQGVDANTTVNTKGITFTGDTGTTERKLGETLAINGDNKLINTAVKDGQVSVSATKALTDAVTNATSALQSIVTTADSEDAQTVNKDSNKANFISGTNIQLTPSKDGITVATKESVDFTTVNATTVNATTVNGNTIKSGDVVITKDGINAGNQTISNVKDGTISENSKEAVNGSQLYTTNQNVTNNTANITKNATDIAKGTAYEGDHAQATTNEFKRALGEKTKVVGGAAKDDLSDNNIGVVSNGVDTLTVKLAKTLEGLTSTTFGNDVNDQTVINKDGVTITNGTEPNKTISLTESGLNNGGQKITNVAKGEKDSDAVNVSQLKEYVGDNSYNWTISDGTKNEAVADNGKVSVKGSANDNSATTPGIVTTLTGTDITVDLSTKTKDDIANGQKHSSVKGDSNVVVNVNTTPNDAGGVQYDVKLADTIVLGQGNNSVTIDGKTGTVSGLNNITWDPKGVYNSGKAATEEQLKLVSDVVQNGWNIQANADVASKVVPGATVQFIDGKNINITRDGNNITIATAANVVTTETDKYVTSGKVDYDTQGNGTTTLTVKNGPDAQISGVKNNFVTSAVTDPDGKKATLTRNDGGTVEINLTDTVNQAVTEATEKGTKYAGDVGNSFTRQLGEVTNVKGGATDNLSDNNIGVVSDGKDTLTVKLAKTLTDLTSAEFGEKDSSDKTVINKDGVSSEVIGKDGNVINSTDLTSDGVTSESVIPETQTSSTETADDGTTTQTDTATDGLDSNEVSSQGMTVSTSTTEVKTTTVTPKDGTTGPKITTVETDTVKTTGVDNDGIRINTTKIVTTTTPDGPTTVVENGEVSLTSGGLNNGGNRAINIAAGVNATDAVNVSQLEAAKTHYYSVKTTTEQANFNNDTATGENSIAAGPNVWAQGVQSAVFGSMAGASSDKATAVGNDAWALGVQSTAIGSGVVAGGKGSVVIGSAENTEKTVSTAEAENSVAIGTHAVVTKADAVAIGHNSGAVNVNTTAIGSGANASGEHSTALGNKASSSGNNATALGVEANASGQESNVLGYKANSNGYRAMALGTLSNAAGTMAIALGSQSNASGIKAQAFGSNAIASAEDAIAMGSNANANAVSGVALGNRSMASGVSGVALGDGAASNAQQSISIGKSARTTATNSIALGNASLATADSAIAMGNSSSAVGSNGIAVGAKSKALKDNAIALGNNATVTANNALALGLNSVAYSDNAIVLGTTSKATGSHAVAIGTGASAYRQNSIAMGKDSSTGGDFAVALGDSANAAAENTLALGKNAVADKKDSVALGNNAYTGDVIATESATLAGQVYEFAGKAPIGTVSVGNQGNERTITNVAAGRISSTSTDAINGSQLYAVSEVASRGWNIQANGDKASQVAPGASVQFIDGTNIDITRDGNNITIATAANVVTTDTDKYVTGGKVAYDNQGNGTTTLTLKDGTEAQVTGAKNNFVTKSETATDGKKATLTRNDGGTVDIDLTNTVNQAVTEATDKGTKYAGDKADTGAAANEFGRKLGETTNVKGGAQGELSDNNIGVVSNGVDTLTVKLAKALSGLTSATFGSDATDQTVINKDGVTINSTTPDKTVSLTETGLNNGGNKITNVAEGKAGTDAVNVNQLNQAITNNAYNWNISDGTNNTAVPDSGTVAVKGSANADSANTVGVVTELEGTNVKVDLSQKAKDDIANGQKHSSVAGDTNFVVTQTTTNAEGGKQYDVKLADTVVIGKDKTNGGNPVTINGTTGEISGLTNTTWDPDTKYEGGKAATQEQLKSVSDVAQKGWNIQANKDTATKVAPGDTVKFIDGENIKITRTGNDITVATAKDVKFDSVKVGDKVSINNDGINAGDTKVTNVTNGTLAADSKDAVNGSQLYATNQNVTNNAANITKNADNIAKGTVYAGDRLDATVTGKTNNFTRALGEQTNVVGGATGELSDNNLAVISNGTDTLTVKLAKSLTDLTNATFGSTDTDKTVINKDGVTVSSDKPEKEVSLTDKGLNNGNNQITNVTSGLVKRDGNPVELSKAEGDVLTNAVNVGDLKTTVTNLTEEGKGGGFGLTAEDNKDVKADLGKTVKVQGDGSVKTTVVEKDGQSALQVGLTENVTVGGQDKPGTITVKGENGKDGVSINGKDGSIGLTGAPGKDGQDAQATIKVVDGTKGLDGNNGKDGESKTRIVYEKPNGGGTEEIATLNDGLNFVGDKGQVIRKKLNDTLAIKGNLDAAAVVTDKNLRVDNDKDQNGELIIKMAKSLTDLTNATFSSDDSDTVIGGNGLTITPKAGDEVSLTDKGLNNGNNTIINVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVTLNGKDGAIGINGKDGSNGSITVKQGKPGVDGKDGETKTRIVYETKDETGKPTTEEVATLKDGLKFVGDTGEVIAKKLNETLAIKGNLTATAAVTDKNLRVDNENGQLIVKMAKSLTDLTNATFGSDNSNTTIGGNGVTITPKGGDASNTVSLTDKGLNNGNNQVTNVSTGLKDRDGNNVTLANASGDVLNNAVNVGDLKDSVNNLTNATTGGFGLTDEKGNDVKADLGKTVTVQGDGSVRTTVVEVEKDGKKEKVLEVGLTNNVTVGNDKEPGTITVKGENGKDGVSISGKDGIGIKGENGQDAVSINGKDGDGTVAVKGKDGKTGVALNGKDGTIGINGKDGSNGSITVAQGEKGLDGNDGANGKTKTRIVYEKPNGDKEEVATLNDGLNFVGNDGKVIAKKLNKTLAIKGNLSTAVKDVTANNLRVDNVGDELIINMAKALTDLTSATFTNKDGNKSVVDGNGLTITPTKGGNIVSLTTSGLDNGGNKVINVAAGDVNANSTDAVNGSQLYAVSEVANKGWNIQTNGNDTTNVKPGDTVNFVNGKNIEINNDGTNVTVGLAKDVDLGKDGSIKAGDTIMNNEGVKVGDNVSLTKDGLKAGNVTISATTGINAGDKQITNVASGLGGKKLSEAKGDTLTNAANIGDLQTAVSSVTDASKGGGFGLADDKGANVTQNLGKTIAVKGDGKNISTVVKGGALTVNLNKNVDLGKDGSVTTGAIQKYLIKV

SEQ ID NO: 9 - protein, 2546 aa, Actinobacillus pleuropneumoniae
MNKIFKVIWSHATQTFVVVSELTRSKSKAKASVSDVKKNEVSSLITSGFKLSAISALLISTIPTAYAAVSIGKAEVEKDNKYIVSQGTAANHGQNAIVIGEYAGNQRADDYISDAPNENEKKLNAYNKQKVSGNDLSNTVALGTYAYAWGHDSVAIGTNSQAVHDVQTDNAEFNSRNSVNAGAIAIGFQAIARGDQTVSLGSRAAAYNRQATAIGNDSYAMGVGSVVIGGDDSGYAYESINGKDKRPWANNAGNLGYTNLATGFNPSKEPNKADYRPSAASGNGATVVGVHSQALSKGSTALGVAATAGDGGKTSSEIDKGQIVGIDSTQNIEATAIGALAHARDRNTTAVGHAAKAYSEYSTSIGDDAVAEGKNSSAVGKSSYAKGDYSVAIGNAENTTNATEVNKSKVIANGTRAIAIGTTARTNADDTIAFGTRTESTAGGAISIGYEAKSKALDAIAIGKGAKALNTNTISIGTGNTVNGANSGAIGDPTVVNGTNSYSVGNDNTIDSNNTFVLGNSVTLNKNAKNSTVIGNQNTINLTDVATNNVNTGYVNIQGDHNTINAARGIGILGNENTVDGVTHDVFILGNNVKKTANDSVFLGSGSGYVAEDTTTKGLSKIYTSDTVNGKPVNFAGGNVTAGVVSVGGVNEKDEKSTRRIQNVAPGLISAQSTDAINGSQLYAVADALKTNIPESKWNIKENATQKDVVNSGDNVSFANGNGTTANVSVSQDETTSTVKYSVNQSNLNVAQDGEVTAANKGDNFATADQVAQAINNSEKTTVINNGTTITATPTTTGTVTTYDLDLSKTTKDDIQKGVDAHTTVTTKGLTFTADAGTPTERKLGETLAINGDATLINTKVEDGKVSVAATEKLKTAVTNAESALQSIITTANGVDAQTVNKNSNKANFTNGKNIVLMPSTTGIEVATKDEVDFTTVNATTLNGTTIKSGDVVITKDGINAGDKKITHVADGNVTNDSKEAVNGSQLYATNQNVTNNTNNIAKNTADIAKGTVYAGDYAKDDSVKNEFTQALGNQTNVVGGASKDDLSDNNIGVVSDGVNTLTVKLAKTLEGLTSATFGSDATDQTVINKDGVTINSTTPDKTVSLTETGLNNGGNKITNVAEGKAGTDAVNVDQLTKAITDSAYNWNISDGTNNTAVPDSGTVAVKGSANADSVNTVGVVTELEGTNVKVDLSQKAKDDIANGQKHSSVAGDTNFVVTQTTTNPEGGKQYDIKLADKVVIGKDKPVTIDGTNGTVSGLTNTTWDPNTTYTGGQAATQEQLKSVSDVVQNGWNIQANTDTATKVAPGDTVKFIDGENIKITRAGNDITVATAKDVKFDSVKVGDKVSINNDGINAGDTKVTNVTNGTLAADSKDAVNGSQLYATNQNVTNNAANITKNADNIAKGTVYAGDRLDAAVTGKTNNFTRALGEQTNVVGGATGELSDNNLAVISNGTDTLTVKLAKSLTDLTNATFGSTDTDKTVINKDGVTISSDKPEKEVSLTDKGLNNGNNQITNVTSGLVKRDGSSVELSKAEGDVLTNAVNVGDLKTTVTNLTEEGKGGGFGLTAEDNKDVKADLGKTVKVQGDGSVKTTIVEKDGQSALQVGLTENVTVGGQDKPGAITVKGENGKDGVAINGKDGSIGLTGAPGKDGKDAQATIKVVDGTKGLDGNNGKDGESKTRIVYEKPNGGGTEEIATLNDGLNFVGDKGQVIRKKLNETLAIKGNLDVNATVTDKNLRVDNDKDQNGELIIKMAKSLTDLTNATFSSDDSNTVIGGNGLTITPKTGDEVSLTDKGLNNGNNTITNVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVTLNGKDGAIGINGKDGSNGSITVKQGKPGVDGKDGETKTRIVYNTTTPDGKPVTEEVATLKDGLKFVGNDGKVVTKELNETLAIKGGINTEAGLTAASDRNVGVRENEKGLNIVIAERPTFSGITVDGKDGKDAEVKFAKDGKDGMSIVGTRGADGQNGLTLKGANGKDGVSFKEDGRITNVADGKDGKDAVNKDQLERVNATANAGWKLTINNGNNQTTVTPNATVDLANTDGNIVITKVGNNVNFGLNNTLTVGNDNKPGTMTVKGENGKDGVSISGKDGIGIKGENGQDAVSINGKDGNGAIAVNGKDGKTGGGLDGANGTIGINGKDGSNGTITLAKGEPGVDGKDGKTRIVYETKTPDGKTVTEEVATLKDGLKFVGNDGKVITKELNETLTIKGNLSTAADVTDKNLRVDNVDNALIIKMARTLTDLTNATFTNAGGDKSVVDGNGLTITPSGNASNTVSLTTTGLNNGDNKVINVAAGDVNANSTDAVNGSQLYAVSEVANKGWNIQTNGSNTTNVKPGDTVNFANGKNIEINNDGTNVTVGLAKDVDLGKDGSIKAGDTFVNKDGVKVGDNVSLTKDGLKAGNVTISATTGINAGDKQITNVASGLGGKKLSEAEGDTLMRRISATYKRRYRA

SEQ ID NO: 10 - protein, 2233 aa, Actinobacillus pleuropneumoniae
MNKIFKVIWSHVTQTFVVVSELTRSKSKAKASVSDVKMNDVSSLITSGFKLSLISSLLISSIPRAYAAIAIDSTNNLGNAVNNGTAKANAKNSHQAYNYQNPGNMYYEGQSDTSRYSGTLEASSGIAIGLNATTIASDNYSSGVAIGDNAMATGGLSFALGAYTQATAIGSTVIGTAGLASGFNSLAMMRQSAATNNYAMAIGTASWADGVASLAMGSSATARGKQSIAIGSSDMLAQNGGNGRALTKYDGTNNTQASGVRAIAIGTTARTSADDTIAIGTKTRILEKESGSIAIGNTAGTQGIKDTRADYLGYTPITIKDKVGNQQDKIAIGTNSYADGHDSIAIGTRAQAIFTNNAPSIGTSRNTENAGAVSIGYQSIAQGDQAVALGSRAEALNRQAMALGNDAFASGVGSIVIGGDDSLPHGSADTAGYQLTTGYNPNSAKFRPSAATGNGAVVVGVHSQALSQGSTAIGVAATAGDNDEVKTKDTVKTATAAKEATAVGAKSRAKSEHTTAVGYEAKAFGLNSTTVGAESTAESTNSFAGGYKATANGTNATAIGSSAQTKGDDAIAIGTGAKALNTNTISIGTGNTVTGANSTAIGDPTTISGTGSHSLGNDNIIPDDNSTVIGNSNVLSKGDWSSTSDGSHVVGNNVQIRGENSLAFGNNAMVGDANNEGVSGSVNATAIGTSATAKGANALAVGTGAKASKTNNTAIGASATVTKDAGEDNIAFGSGATVTRGKNNVVIGKGSQADSLNGSSIAIGDGATIGNGVTGNGYAIGTGANIASTNGGIAFGNGAKITNSNGGDGSSVAIGEGAVATGTRAISLGHKANTDGNQVDSIAIGSSAKTSGNNNIAIGNNATAQGNQAEQIVIGTGANASDTSQYSIVMGSGAGATREHSTVLGSKANSIVDGGVALGANSVSDRQDGGSATGHRSTYEPYIPATADQAQKDAINATKGTTGAVSVGSSTVKRQITNLAAGAEDTDAVNVAQLKAAVTTAVASSTWNIKENTTQKDVVNSGDNVSFANGVGTTATVEVTDSDKTSTVKYSVNKSNLNVATDGTVTAANTGDNFATAEQVAQAINNSEKTTAINNSTTITAKPITVGTVTTYDLDLTQETKDKLQQGVDANTTVNTKGITFTADNAGAKTERKLGETLAINGDKTLINTTVEAGKVSIAATDKLKTAVTNAESALQEIVTTVNGTTAQTVNKNSNKANFINGKNIELTPSKDGITVATKENVDFTTVNATTVNATTVNGNTIKSGNVVINQDGINAGGQTITNVKAGDKGTDAVNVNQLNEVKNTANAGWTIQANGQNGSSVKPNETVSLNNADNNIVISKEASNDNVTFALNSTLNIGNTKPIKIDGTTGTISGLVSTLPNSSTLTAQTRPTTTETILSFAATVGDVLNSGWNLQGNSQAVDFVSAYDTVNFVSGSGTTAEVTVDAGKAVSTVKYSVNKSDLSVTGGTVTAGKAGDNFATADQVAKAINDSEKTSSVVSTTQTIKITPKVSGNNTEYDLDLSDATKTSLQKADSALQSWTAQANGQDVKTVSKDNATLNFVNGTNIQVTNDKGQIQIATVDAPTFTTVTAGTVNAENFTTGTVSITDNGINAGNQKITNLANGTANSDAVTLAQLNASKSVVQAGNNTHVRSETVTDGSTIYTVDANATTVSGSNALNITKSDAGNNVTNYALDLSDKTKGEIKQGVDANTTVNTKGITFTGDTGTTERKLGETLAINGDNKLINTAVKDGQVSVSATKALTYAVTNATSALQSIVTTADSKDAQTVNKDSNKANFISGTNIQLTPSKDGITVATKENVDFTTVNATTVNATTVNGDTIKSGDVVITKDGINAGNQTISNVKDGTISENSKEAVNGSQLYATNQNVTNNTNNIAKNTADIAKGTVYAGDVGNSFTRPLGETTNVKGGATDNLSDNNIGVVSDGKDTLTVKLAKTLTDLTSAEFGEKDSSDKTVINKDGVSSEVIGKDGNVISSTDLTSDGVTSESVIPETQTSSTETADDGTTTQTDTTTDGLDSNAVSSQGMIVSTSTTETKTTAVTPKDGTTGPKITTVETDTVKTTGVDNDGIRINTTKIVTTTTPDGPTTVVENGEVSLTSGGLNNGGNRAINIAAGVNATDAVNVSQLEAAKTHYYSVKTTTEQANFNNDTATGENSIAAGPNVWAQGVQSAVFGSMAGASSDKATAVGNDAWALGVSIYGNRFRCCCGRKRLCCYR

SEQ ID NO: 11 - protein, 1383 aa, Mannheimia haemolytica
MNKIYRIVWNHAKRTWIVASELSRSANKSNATNTSLTTNIIKLSTLSLALSAGFASAATYSPTYGTIPDNNPQTSIAIGDGSKVNSTLVSTITGLEGNSGKVSTALGYNTTATADFSTALGGFANASFASTAVGGQATADKRSVAVGYNATAMGLREVFIGDHAGLNHSNSTEYNIGIGYSASSNVTGNNTISIGNTAGDGTSGSHNIAIGTYANAKLAGPTTNVTSDDNIAIGNSALANGVNNYKTTAATGETVIGKATAVGSHANATGIVSSAYGAEANATAIHSTAIGAKSQANGDNSTAIGYEAKTSGHGATSLGYYANATANLTTAVGTNAGATSDYASAFGREANASGGSATALGNRATASGAASVALGVSAKATNQRTIAIGESSNASAFNATAIGRNATAEHTDSIALGSNSVTAIAIPTTNATVNGITYSDFAGTNPIATVSIGAEGKERTITNVAAGRISLSSTDTINGSQLYLTQQAIGNVAATTANILGGGAAVTENGNITFPTYALVNGTPDADKEGKQGRYTTVSAALSALNTAVISPLTFAGDTGTNFERHLGSTVKIKGGSTGILTENNIGVVADGNSTLTIKLAEKVNLGANGSLTTGDTVVNNTGITIANGVADKPVSLTKSGLDNGGNKIANVAAGDVDTDAVNVSQLKQAISKFATHYVSISDDGIQRANYDNSGSSGVNPMAIGVATSANGELATALGSEAEANGERTTAVGPRATADGMNATSIGYNANANATNALAVGSAANANADTSTAIGTASTATATRATALGSKSEATGENSTAVGYEASSIGADSLAAGYNANASGTQSTALGNSANAGGIWSTSVGRNANAAGSSAIALGNSANAAGVASIALGVSSQATTTAAVALGQNAKATHQGSVALGTNSETVATVATKSATLNGNTYTFAGTTPSSTVSIGSVGNERTLTNVAAGRILDSSTDAINGSQLYAAYTEIDGLNTKVNELSNGALTFVDDAGTEIVRKLGTSLNVKGGADATILTDNNIGVVATDANTLTVKLAKDIDLTPAGSVAVGNSKLNNNGLTINNGPSVTMTGVDAGKLKITNVADGDISPISADAVNGSQLYDTANTIATALGGNSSVNANGAVSAPSYTVVDGAPTNEVSKTVNNVGSAITALNDAVTSPLTFAGDTGTPSQRKLGSTVTVKGGVSNESQLTDNNIGVISNGNGSLTVKLAKDIKVNSVTAQTVTANIAVADTVKTGDTTIDTNGLTIVGGPSITKTGINAAGTKVTNVKAGTEDTDAVNFSQLKATEKNINNKINNIDSKVNKVDKRLRAGIAGATATAGLPQAYLPGKSMLATAGDTYRNEAAIAVGYSRISDNGKVIYKLTGNSNTRGDFGGSIGMGYQW

SEQ ID NO: 12 - protein, 2023 aa, Mannheimia haemolytica
MNKIFKVIFNQTTQKMEVVSELARSQGKAASSTDKRGTIKLAAASILGGATLISGMAAFMLSATPAEAAVIIDRIEVQQKAPKKVVNGGIDIYTNTSLATDSITNPYNYFNPGSLSYADRTKLQESNNSVNKEKDSRLLYKSNTTSAIVIGHNAVALDNNRKSRGKPKASGIAIGDYSYSGGGLSIAIGSFSRTASIGSVALGTSARAEGFNSFAAMRQSAAIADYSTAIGSTSWANATASFAIGASATALGDRSFAIGSGTPQTVDGSYGHENRRSIYDGQNNTLAFGTDAFSIGTKAKTNGNHSFAIGTNAWSGGFKLENSNISELGQVINPDTAKVANNAFALGTNTRANSNDTLAFSTNASATGDGSIAFGVNSNTTGIGAIAFGNASKAWKSNAISIGWNANSHAEKVISIGSHSNASKDGAMAIGDTANSSGNNSVAIGYSANVSKANATAVGTRANITHEGGTAIGFEAKSKGNYATAIAYNSTAKGKNSIAAGVNSTVSGIRAAALGYNNTVSGKDTFVLGSNVTVKTKGSVILGSDSTEATATKEDTATVGTLKYSSFAGDENVVDGDYVSVGNATNARQIKFVAPGKIASNSTDAINGSQLYAVADTLVKTGFNITADHSGLGGGAKEDNVKLGETVKYTSTDKNVVTTVTNNTIDFGLSDNISVGGKPGKDGSVGVKGADGKDGVTIKPEAIVFHGVDGVNGKDGKDGKASIKVEKGAKGLDGNDGKDGESKTRIVYEKPNGDKEEVATLNDGLKFGANDGKVHDAKLNTQVDIKGATTNTDWAKFDAGKNIMTQVVGNTITVALAKDLAGLDSATFGTQGDSTVINKDGVTIAKKDPNDATKSNVTLTEDGLSNGGNQITNVDSGLKGKDGQPVKLTEAAGDVLNNAVNVGDLKNTVTETVTEVTTDAFGLKDENNKEFKQNLGTTAKIIGDGNVKTNVVEKDGGHALEVSLVNNITVGKDGEPGKVGIAGKDGKDAIALDGKDGAITVGRPGKDGIDGTIGVNGKDGSAVVLNGKDGSIGLNGTNGKDGLTIKGTEGKPGLVGKDGLPGKDGVTRIQYQPTDGNGDPIGEPEQVATLNDGLHFKGDDATLVSKKLNETLEIIGGADKDKLTANNIGVNSKDGKLSVQLAKDLKDLNSATFKPQDGENGQEGKGPTTVVNNNGVTITPKDGTDGKAVKLTEDGLDNGGNQIVNVKSGIDGLAGKDGINGKDGKPITSVNDLTDDQLKEVGTNAANINDVVNASNDIIEKGFSIAADNGETDHVKLGETVKYTSKDKNIITTVSDNVIDFTLNNDFTIGGPGKDGSIGVKGADGTVGVTLNGKDGSIGLTGPKGADGKDGASATISVKDGAKGLDGNDGKDGESKTRIVYEKPDGTTEEVATLNDGLRFVGDDGKEIVKKLNETLKISGGDTDLTNLSDNNIGVVNNNGALTVKLAKDIDLTKDGSVTIGDTTVNNDGLTIKDGPSVTKAGINAGDKKIINVQDGEISATSKDAVNGSQLYQVQEVANAGWNLTANGKDKGNVKPGATVDLNNKDGNIIITKEGNNVTFGLKNNLTIGGKDGKDGQIGIAGKDGKDGVTIKGDGTITAGRDGKDGVDGSIGATGKDGASVVLNGKDGSIGLTGPKGADGKDGASANIAVKDGAEGVDGTNGKDGLPGENGKTRIVYETKDKDGKTVTEQVATLNDGLIFSGNNEDVKNRQKLNTEVKVKGEGVDKTASENFKSASGNINVKANGTDTLEVQLAKAIDLTEDGSVTTGDTTVNNNGLTIKDGPSVTKGGIDAKGTKITNVKDGDVTATSKDVVSGSQLYNAMQNTGWYLTVDGNVAETSEKGAQRVNNNGKVAVSGGKNIVVSRKGSTVEVATSSNLEFDSVKVGGTTISSTVAKDGVNELNIAGSKNAQTRITNVAPGVKGTDAVNVNQLKGAENRLNNRISNVDKDLRAGIAGALAAGNLYHVTQPGKSMVSAGVGAYRGQGAIAVGYSRLSDNGKVGIKFSVNSNSRGDTGAAASVGYQW

SEQ ID NO: 13 - protein, 719 aa, Mannheimia haemolytica
MGARATSANAIALGTDTAATGNRATAFGAGALATGNRSTVMGWRSAASGTRSFAMGSGAMGDKSVANATYSLALGSSANANATSTATRGTAIGYNATVSGVNATALGVSAVSSNQSTVAIGDSAKASAGFAIALGSGANVALNNSVALGSNSTVSNAVAVPSATVNGITYSGFAANTTAAGNVVSVGSNTIKRQIQNVAAGQISATSTDAINSSQLYMAMNATGNLANSTKNILGGNATVKPDGSVTYTNIGGTNKNTIEEALKAVKTEVVAGSNVNITNATGANGQTIYTVNAYNTTANSSSPDYITVTGKAATAANTTNYEIGLTKKAIDDFTKDTQATVVSNDGTVTVKSTERNANGTVIYDLSVNIPAQASQIQYFSVNSTVPENQANDGAKSRNSIAIGPNATATGGEQAAVALGTNSNANGNGALSLGVATVSKGIQATAVGHSANATANGTTALGRQTNATAGDATAVGSNANATAEKASAFGVAANASANASLAVGANSIASAQSAVAVGTRANATAQFATALGMGAQATLNSSVALGSESVVRAATPTENATVGGITYNGFAGVNKDTNYVVSVGSAGKERQIQNVAAGQISATSTDAINGSQLYMAMNATSNLANSTANNFGGGSVVNPDGSVTQPKYNVTNAAGTQYGNTATNVGDAITNLNNYVNQGFNIKDNAGETKGTVTPNESVQFVNGKGTVSNVTQEADGVK

SEQ ID NO: 14 - protein, 877 aa, Mannheimia haemolytica
MRKELVNRRNQLTELENNFNTSFLPEKNKQEAYQKFSTVIESLDWNKLSATNGVDQLATDLKTKIETDFPNINFSNDQYKDLIKGYRNIQGNLVTAIPKFLNKVNDDFAISNSDRGLYYNYLPYHGIDRWEGSNFNFRTLNGVYYDDNLTSTELLRKFMIQRDYNTLSELSRAHAGNADGLAKNYHLVVAKMSYENLKKELSKNSDSKFYKLKKLYEAYGEFSNLNEYVSSSFQGNPLELLTVPVLKKSLENQELLPNKLYRKFNIDNQAIAQTTSNITKEEIADLEKTVENFNKFKELIDYSSNAWIFNKEEYRTYMNDNVIPFMEKVEEFTKTLKTLSREDLSEEERTAKTIDSVKLRIYLNEQARNRKNYINGFVPTDWISEITDQMTKSKNLWLKYEEESKTTLKPYQEETISSLIKDEINKKMAEIDTTTLEIANKERELEELENQINAFTLTDEEKLAEDVKNDLISKLDEAKAALAKNQQTLKEKHDELLGLNNKLDSSPLVKNGKNALAEGTNAFASGENAIAFGTDSQATGNNAIALGANSKANAESAIAIGKGAQALKEKALALGENAIANGASAIAIGDNIGVSGEKAVGIGSNAIVSGNGAISIGSDNRVSHKNAVAIGSNITQTAENSVNLGNESATTVELTAETAGTTQYAHSEILGEIYKFAASEPAGVVTVGAKGKERRIQNVAAGLVSETSTDAVNGSQLYAVAKAANEGKIGVVRYTNTDGVVRIANHLDGNEVNISNKSGNTRLLTGLSEGLAPTDAVNKAQLDREIGVVNNKISAVEKEVKKVKGSVSNAIAIASLPQVSVPGKRQLSVATGHTLGTTSVAVGLNGLSDNGRISYKLGTSVSQNSNFAVGAGIGFSW

SEQ ID NO: 15 - protein, 814 aa, Mannheimia haemolytica
MASREEVKSTDKSVTVNTTKNADGANVFDLSVNTDDVTIVKDPTTGAIKANTTALNDANNDGRIDEPTADDAKKLVTAGDITNAINNSGFTLKTSAVEGGEKLSGGDELINPGKAVEMVAGKNLTVKQEADGKVIYATKDDVKFSSVTSNTVTVPTDEADPANNPITINKDGINAGNKAISNVASNLTPVTADDKVQPADNNPTNLADKLSNAATLGDVLNAGWNLQGNGKAVDTVVHNDTVDFINGKGTTVTVENKDGKNTIKVDSPIEFVNQDPTDSSTPSNTAKFTGEAPVQLGNVASSVRNEDGSTPEGKDRAEAIKNAEGDKLNNVVNLGDLQAATNAATTKVGGNRGVTITPSTNADGSTTYNVEAKTDGTTIKVDNEGNITANTSELGNNEDGTVKAPTQPNALVTAQTVADAVNNAGFNIKSAGNKAAGDQAATKLVKTGEEVVFEAGDNLTVKRDGNQFTFATAKDVSFNSVQFSENGPKITNDGDNIKVGDKDGKPTKITNVADGDISPVSTDVINGKQLNNYAKVNGNNIGTDEDGSINIVNGNGTTITSDKAGEVKVNVNSTDLTVADNGKINVQDPNGTGSHFVNATTVANAVNNVSWNVDSKAVGTGVVEGDKAPAKVKAGSTVSVNAGNNIKVTRKGSDVTVAVSDTPEFTSVKTGDTLVNNNGVTINNGSAGKAVSLTKDGLNNGGNRITNVKAGEADTDAVNVGQLKGAVNHLNNKIHRNNREARAGIAGSNAAALPQVYIPGKSMVAAAGGTFKGENALAVGYSRSSDNGKLILKLQGNANSRGDFGGGVGVGYQW

SEQ ID NO: 16 - protein, 651 aa, Actinobacillus pleuropneumoniae
ATLKDGLKFVGNDGKVITKELNETLTIKGNLSTAADVTDKNLRVDNVDNALIIKMARTLTDLTSATFTNAGGDKSVVDGNGLTITPTKGGNTVSLTTSGLDNGGNKVINVAAGDVNANSTDAVNGSQLYAVSEVANKGWNIQTNGNDTTNVKPGDTVNFVNGDNIAITNDGTKVTVGLVKNVDLGEDGSIKAGDTFVNKDGVKVGDNVSLTKDGLTAGDVKISATTGINAGDKQITNVASGLGGKKLSEAEGDTLTNAANIGDLQTAVSSVTDASQGGGFGLADDKGANVTQNLGKTIAVKGDGKNISTVVKGGALTVNLNKDVDLGKDGSLTIGNTTINSDQVKVGDVTVSSNGKVSGVADGDISPNSTEAINGSQLYDANQNIANYLGGGSKLDDKGNVIAPTYTITKVDGNTTTANNVGDAITNLNNEVVKPLTFEGDTGVASKRKLGSTVTIKGGVQDSSKLSENNIGVVSDGKGTLAVKLAKDIKVDSVEAKTVNANTVNANTVKAGDTTINSDGVTIKDGPSVTKSGINAAGNRITNVKAGQADTDAVNVSQLKGAVGHVNQRINKVNKDLRAGIAGANAAAGLPQAYMPGKSMMAVAAGTYKNESALAVGYSRSSDNGKVILKLQGNANTRGDLGGSVGVGYQW

SEQ ID NO: 17 - protein, 924 aa, Actinobacillus pleuropneumoniae
VTKLDGKNITVDLSEKAKQELENGQKHSSVNGDTNVLVEVNKAPNAEGGKQYDVKLADKIVIGQGDNSVTIDGTSGTVSGLNNLTWDPSATYEGGKAATQEQLKLVSDEVQKGWNVQTNNDTAQKVAPGETVKFVDGKNIKVTSNGKEITIATADNVVTTDTDKYVTGGKVEYDNQGNGTTTLTLRDGSTAQVTGAKNNFVTSAKTDPNGKKATLTRNDGGTVDIDLTNTVNQAVNEATEKGTHYEGDKPDTGATANNFKRKLGETTKVLGGAQGELSDNNIGVVSNGSDTLTVKLAKSLTDLTNATFGSNDDKTVINKDGVTITNGADANKTVSLTDGGLNNGGNKITNVAKGDADTDAVNVSQLNQAIKDSSYNWNISDGKTEQAVPDDGKVSVKGSANDDSKATSGIVTTLTGTDITVDLSDKSKQDIADGKKHSSVEGDANVVVTQTTTNKDGGKQYDVKLADKVSIGSDKATTVTIDGTKGTVSGLTNTTWDPNKEYTGGQAATQEQLKSVSDVANSGWNISANGQNESNVGPKGKVSFNNTDGNVLISKETTDNNVTFNLNNDLTVGGAGKDGKDGKDGTLSVKGADGKTGVTLNGKDGTIGLTGAPGKDGKDAQATIKVVDGTKGLDGNNGKDGESKTRIVYEKPNGGGTEEIATLNDGLNFVGDKGQVIQKKLNETLAIKGNLDAAAVVTDKNLRVDNDKDKNGELIIKMAKSLTDLTNATFSSDDSNTVIGGNGLTITPKAGDEVSLTDKGLNNGNNTIINVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVTLNGKDGSIGLTGPKGADGKDGANATISVVNGPVGVDGTDGKDGKDGMTRIVY

SEQ ID NO: 18 - protein, 2378 aa, Actinobacillus pleuropneumoniae
KDESTTKSGIVTKLDGKNITVDLSEKAKQELENGQKHSSVNGDTNVLVEVNKAPNAEGGKQYDVKLADKIVIGQGDNSVTIDGTSGTVSGLNNLTWDPSATYEGGKAATQEQLKLVSDEVQKGWNVQTNNDTAQKVAPGETVKFVDGKNIKVTSNGKEITIATADNVVTTDTDKYVTGGKVEYDNQGNGTTTLTLRDGSTAQVTGAKNNFVTSAKTDPNGKKATLTRNDGGTVDIDLTNTVNQAVNEATEKGTHYEGDKPDTGATANNFKRKLGETTKVLGGAQGELSDNNIGVVSNGSDTLTVKLAKSLTDLTNATFGSNDDKTVINKDGVTITNGADANKTVSLTDGGLNNGGNKITNVAKGDADTDAVNVSQLNQAIKDSSYNWNISDGKTEQAVPDDGKVSVKGSANDDSKATSGIVTTLTGTDITVDLSDKSKQDIADGKKHSSVEGDANVVVTQTTTNKDGGKQYDVKLADKVSIGSDKATTVTIDGTKGTVSGLTNTTWDPNKEYTGGQAATQEQLKSVSDVANSGWNISANGQNESNVGPKGKVSFNNTDGNVLISKETTDNNVTFNLNNDLTVGGAGKDGKDGKDGTLSVKGADGKTGVTLNGKDGTIGLTGAPGKDGKDAQATIKVVDGTKGLDGNNGKDGESKTRIVYEKPNGGGTEEIATLNDGLNFVGDKGQVIQKKLNETLAIKGNLDAAAVVTDKNLRVDNDKDKNGELIIKMAKSLTDLTNATFSSDDSNTVIGGNGLTITPKAGDEVSLTDKGLNNGNNTIINVAPGVNGTDAVNKDQLDGVNATANAGWNLTTNGDNTNASNVAPNSTVDLANTDGNIVITKAGNNVTFDLNNNLTVGGPGKDGKDGVDGQLGVQGKDGKTGVTLNGKDGSIGLTGPKGADGKDGANATISVVNGPVGVDGTDGKDGKDGMTRIVYTDPKGTTHNVSTLNDGLNFAGNQGDTIVKKLNETLTVKGALANTADASSENLRVDSQDGALVVKLAQNLANLTTATFGNTDTDKTVVSKDGVTISNGSDKDKTVSLTDKGLNNGNNQITNVTSGLTDSTGQKSDLANATTTNAVNVGDLKDTVNNLTNATTGGFGLKDDNNTEVKQDLGKTIQIKGKDGVTVTSNVADKSLEVALQGDVTVNGKDGKDGSIGVKGADGKDGTKITKDAVVFNGVDGKDGKDGQVSIKVEQGEKGIAGNDGANGTTKTRIVYEKPNGDKEQVATLNDGLNFVGDKGQVIQKKLNETLAIKGNLDANATVTDKNLRVDNDKDQNGELIIKMAKSLTDLTNATFSSGDINATIGGNGLTITPKGGDVVSLTDKGLNNGNNTITNVAPGVNGTDAVNKGQLDGVNATANAGWNLTTNGDNTNASNVAPNSTVDLANTDGNIVITKAGNNVTFDLNNNLTVGGPGKDGKDGVDGQLGVQGKDGKTGVALNGKDGAIGINGKDGSNGSITVKQGKPGVDGKDGETKTRIAYETKDETGKPTTEEVATLKDGLKFVGDTGEVIAKKLNETLAIKGNLTATAAVTDKNLRVDNENGQLIVKMAKSLTDLTNATFGSDNSNTTIGGNGVTITPKGGDASNTVSLTDKGLNNGNNQVTNVSTGLKDRDGNNVTLANASGDVLNNAVNVGDLKDSVNNLTNATTGGFGLTDEKGNDVKADLGKTVTVQGDGSVKTEVVEKDGKKALQIGLTNNVTVGNDKEPGTITVKGENGKDGVSISGKDGISIKGENGQDAVSINGKDGNGAIAVNGKDGKTGVGLDGANGTIGINGKDGSNGTITLAKGEPGVDGKDGKTRIVYEAKTPDGKTVTEEVATLKDGLKFVGNDGKVITKELNETLTIKGNLSTAADVTDKNLRVDNVDNALIIKMARTLTDLTSATFTNAGGDKSVVDGNGLTITPTKGGNTVSLTTSGLDNGGNKVINVAAGDVNANSTDAVNGSQLYAVSEVANKGWNIQTNGNDTTNVKPGDTVNFVNGDNIAITNDGTKVTVGLVKNVDLGEDGSIKAGDTFVNKDGVKVGDNVSLTKDGLTAGDVKISATTGINAGDKQITNVASGLGGKKLSEAEGDTLTNAANIGDLQTAVSSVTDASQGGGFGLADDKGANVTQNLGKTIAVKGDGKNISTVVKGGALTVNLNKDVDLGKDGSLTIGNTTINSDQVKVGDVTVSSNGKVSGVADGDISPNSTEAINGSQLYDANQNIANYLGGGSKLDDKGNVIAPTYTITKVDGNTTTANNVGDAITNLNNEVVKPLTFEGDTGVASKRKLGSTVTIKGGVQDSSKLSENNIGVVSDGKGTLAVKLAKDIKVDSVEAKTVNANTVNANTVKAGDTTINSDGVTIKDGPSVTKSGINAAGNRITNVKAGQADTDAVNVSQLKGAV

SEQ ID NO: 19 - protein, 563 aa, Actinobacillus pleuropneumoniae
ATLKDGLKFVGNDGKVITKELNETLTIKGNLSTAADVTDKNLRVDNVDNALIIKMARTLTDLTSATFTNAGGDKSVVDGNGLTITPTKGGNTVSLTTSGLDNGGNKVINVAAGDVNANSTDAVNGSQLYAVSEVANKGWNIQTNGNDTTNVKPGDTVNFVNGDNIAITNDGTKVTVGLVKNVDLGEDGSIKAGDTFVNKDGVKVGDNVSLTKDGLTAGDVKISATTGINAGDKQITNVASGLGGKKLSEAEGDTLTNAANIGDLQTAVSSVTDASQGGGFGLADDKGANVTQNLGKTIAVKGDGKNISTVVKGGALTVNLNKDVDLGKDGSLTIGNTTINSDQVKVGDVTVSSNGKVSGVADGDISPNSTEAINGSQLYDANQNIANYLGGGSKLDDKGNVIAPTYTITKVDGNTTTANNVGDAITNLNNEVVKPLTFEGDTGVASKRKLGSTVTIKGGVQDSSKLSENNIGVVSDGKGTLAVKLAKDIKVDSVEAKTVNANTVNANTVKAGDTTINSDGVTIKDGPSVTKSGINAAGNRITNVKAGQADTDAVNVSQLKGAV

SEQ ID NO: 20 - protein, 6 aa, Artificial Sequence
  Hexapeptide for testing glycosyltransferase activity
DANYTK

SEQ ID NO: 21 - protein, 5 aa, Artificial Sequence
  Substrate peptide for bacterial OST from C. jejuni
DQNAT

SEQ ID NO: 22 - protein, 5 aa, Artificial Sequence
  Substrate peptide for bacterial OST from C. jejuni
DFNVT

SEQ ID NO: 23 - DNA, 29 bp, Artificial Sequence
  Amplification primer
gaaccatgggggataagtctgttgcaaat

SEQ ID NO: 24 - DNA, 64 bp, Artificial Sequence
  Amplification primer
cttctcgagttagtgatgatgatgatgatggtggtggtggtgatccggattcacaacagaaccg

SEQ ID NO: 25 - DNA, 41 bp, Artificial Sequence
  Amplification primer
gaaccatggtaatcaaggctaacactacggcattaaatgat

SEQ ID NO: 26 - DNA, 64 bp, Artificial Sequence
  Amplification primer
gttctcgagttaatgatgatgatgatggtgatgatggtggtgctgcggtaacgctgctgcattt

SEQ ID NO: 27 - DNA, 31 bp, Artificial Sequence
  Amplification primer
cagaattcatgtcagcagaaaatatgcctag

SEQ ID NO: 28 - DNA, 63 bp, Artificial Sequence
  Amplification primer
gttctgcagctacagatcctcttctgagatgagtttttgttcgctcttagtttcggtttttgc

SEQ ID NO: 29 - DNA, 33 bp, Artificial Sequence
  Amplification primer
gaacatatggaaaacgaaaataaaccgaatgta

SEQ ID NO: 30 - DNA, 68 bp, Artificial Sequence
  Amplification primer
cgaattctacagatcctcttctgagatgagtttttgttcgtcgacctcgagattttcttttaggaacg

SEQ ID NO: 31 - DNA, 29 bp, Artificial Sequence
  Amplification primer
catccatggaaaacgaaaataaaccgaat

SEQ ID NO: 32 - DNA, 30 bp, Artificial Sequence
  Amplification primer
cactgcagaattctacagatcctcttctga

SEQ ID NO: 33 - DNA, 35 bp, Artificial Sequence
  Amplification primer
aaaccatggcaacccttaaagatggcttaaaattc

SEQ ID NO: 34 - DNA, 35 bp, Artificial Sequence
  Amplification primer
tttggtaccttataccattgataacctacacctac

SEQ ID NO: 35 - protein, 5 aa, Artificial Sequence
  Consensus sequon for bacterial OST
DQNAT